(19) United States
(12) Patent Application Publication
(10) Pub. No.: US 2023/0330376 A1
(43) Pub. Date: Oct. 19, 2023

(54) RESPIRATORY VALVE APPARATUS AND RELATED METHODS

(71) Applicant: Innovation Lab, LLC, La Palma, CA (US)

(72) Inventors: Michael Reber White, La Habra Heights, CA (US); Jeff Wei-Kay Ho, Los Angeles, CA (US); Omar Abdallah, Los Angeles, CA (US); Jinyu Yang, Los Angeles, CA (US); Amy Sara Lam, Anaheim, CA (US); Gordon Fox Grajek, Orange, CA (US); Hieu Van Le, Fountain Valley, CA (US); Myong Kim, Tustin, CA (US)

(21) Appl. No.: 17/922,563

(22) PCT Filed: Apr. 30, 2021
(86) PCT No.: PCT/US21/30320

§ 371 Date: Oct. 31, 2022

Related U.S. Application Data
(60) Provisional application No. 63/019,043, filed on May 1, 2020.

(51) Int. Cl. A61M 16/04; A61M 16/08

(52) U.S. Cl. CPC A61M 16/0463; A61M 16/0833; A61M 39/223

(57) ABSTRACT

Aspects of the current subject matter can include systems, devices and methods related to embodiments of a respiratory valve apparatus. The respiratory valve apparatus can include a first port configured to couple to a ventilator, a second port configured to couple to a resuscitation bag or a transport ventilator, a third port configured to couple to an endotracheal tube, and a piston within a housing of the respiratory valve apparatus. The piston can include a first flow pathway that allows fluid flow between the first and third ports when in the first position and a second flow pathway that allows fluid flow between the second and third ports when in a second position. The piston can prevent fluid flow between the second and third ports when in the first position and prevent fluid flow between the first and third ports when in the second position.

### CROSS-REFERENCE TO RELATED APPLICATION

This application claims priority under 35 U.S.C. § 119(a) to U.S. Provisional application Ser. No. 63/019,043, filed on May 1, 2020 and entitled “RESPIRATORY VALVE APPARATUS AND RELATED METHODS,” the disclosures of which is incorporated by reference herein in its entirety.

### TECHNICAL FIELD

The subject matter described herein relates to systems and methods relating to various embodiments of a respiratory valve apparatus.

### BACKGROUND

Respiratory support systems are commonly used to support the respiratory system of a critically ill patient for maintaining optimal blood oxygen levels, as well as optimal carbon dioxide levels and acid base balance. Typically, a prior art respiratory support system includes a tracheal tube, positioned either directly through the nose or mouth into the trachea of a patient. A multi-ported manifold is connected to the endotracheal tube at one port position, and a source of breathable gas is connected at a second port. The respiratory support system assists the patient in maintaining adequate blood oxygenation levels without overtaxing the patient's heart and lungs.

While a patient is attached to the respiratory support system, it is periodically necessary to aspirate fluids and or secretions from the patient's trachea and lungs. Aspiration and positive pressure ventilation has been accomplished by disassembling part of the respiratory support system, either by removing the ventilator manifold or by opening a port thereof and inserting a small diameter suction tube down the tracheal tube and into the patient's trachea and lungs. The fluid was then suctioned from the patient and the suction catheter was removed and the respiratory support system reassembled. However, due to the interruption of respiratory support during this procedure, a patient's blood oxygen level can often drop and the carbon dioxide can change to unacceptable levels. Disassembly of the respiratory support system for suctioning and other invasive procedures, such as bronchoscopy, can expose the patient's lungs to possible contaminants in the environment thereby increasing the chances of acquiring ventilator associated pneumonia (VAP). Additionally, unless a sufficient positive end expiratory pressure (PEEP) level is maintained, then the lungs can collapse. This creates a dangerous condition for the patient because the lungs can be difficult, and sometimes impossible, to reinflate.

### SUMMARY

Aspects of the current subject matter can include systems, devices and methods related to various embodiments of a respiratory valve apparatus. In one aspect, a respiratory valve apparatus can include a first port configured to releasably couple to a ventilator, a second port configured to releasably couple to a resuscitation bag or a transport ventilator, a third port configured to releasably couple to an endotracheal tube, and a piston slidably disposed within a housing of the respiratory valve apparatus. The piston can be biased in a first position within the housing. The piston can include a first flow pathway that allows fluid flow between the first port and the third port when the piston is in the first position and a second flow pathway that allows fluid flow between the second port and the third port when the piston is in a second position. The piston can prevent fluid flow between the second port and the third port when in the first position and prevent fluid flow between the first port and the third port when in the second position.

In some variations one or more of the following features can optionally be included in any feasible combination. The respiratory valve apparatus can include can also include a spring that biases the piston in the first position. The spring can be compressible to thereby allow the piston to move into a second position. The piston can be positioned adjacent the second port when in the first position for allowing the resuscitation bag or transport ventilator to move the piston into the second positon as a result of coupling the resuscitation bag or transport ventilator to the second port. The movement of the piston into the second position can prevent fluid flow through the first port and allow fluid flow through and between the second port and the third port.

In some embodiments, the respiratory valve apparatus can further include a first conduit including the first port at a first end and a first connection member at a second end, and the first connection member can be coupled to the housing and allow rotational movement of the first conduit relative to the housing. The respiratory valve apparatus can further include a second conduit including the third port and a forth port configured to releasably couple an accessory device. The accessory device can include one or more of a suction catheter, a bronchoscope, and a drug delivery catheter. The second conduit can further include a second connection member configured to couple to the housing and allow rotational movement of the second conduit relative to the housing. The fourth port can include a sealing member that prevents fluid flow therethrough when the accessory device is uncoupled from the fourth port.

In another aspect, a respiratory valve apparatus can include a first port configured to releasably couple to a ventilator, a second port configured to releasably couple to a resuscitation bag or a transport ventilator, a third port configured to releasably couple to an endotracheal tube, and a lever member pivotably disposed within a flow pathway extending along a housing of the respiratory valve apparatus. The lever member can be biased in a first position to allow fluid flow between the first port and the third port, and the lever member can be positioned adjacent the second port. The lever member can pivot into a second position as a result of coupling the resuscitation bag or the transport ventilator to the second port thereby allowing fluid flow between the second port and the third port.

In some embodiments, the lever member can prevent fluid flow between the second port and the third port when the lever member is in the first position, and the lever member can prevent fluid flow between the first port and the third port when the lever member is in the second position.

In yet another aspect, a modular respiratory valve apparatus can include an airflow module including a first port configured to releasably couple to a ventilator, a second port configured to releasably couple to a resuscitation bag or a transport ventilator, and a first housing connection port. The modular respiratory valve apparatus can further include a procedure module including a third port configured to releasably couple to an endotracheal tube and a second housing connection port configured to releasably couple to the first housing connection port. The modular respiratory valve apparatus can further include a piston slidably disposed within the airflow module, and the piston can be biased in a first position within the airflow module. The piston can include a first flow pathway that allows fluid flow between the first port and the third port when the piston is in the first position and a second flow pathway that allows fluid flow between the second port and the third port when the piston is in a second position. The piston can prevent fluid flow between the airflow module and the procedure module when in the second position and allow fluid flow between the first port and the third port when in the first position.

In some variations one or more of the following features can optionally be included in any feasible combination. The modular respiratory valve can further include a spring that biases the piston in the first position. The spring can be compressible to thereby allow the piston to move into a second position. The piston can be positioned adjacent the second port when in the first position for allowing the resuscitation bag or transport ventilator to move the piston into the second positon as a result of coupling the resuscitation bag or transport ventilator to the second port. The movement of the piston into the second position can prevent fluid flow through the first port and allow fluid flow through and between the second port and the third port.

In some embodiments, the modular respiratory valve apparatus can further include a first conduit including the first port at a first end and a first connection member at a second end, and the first connection member can be coupled to the housing and allow rotational movement of the first conduit relative to the housing. The modular respiratory valve apparatus can further include a second conduit including the third port and a forth port configured to releasably couple an accessory device. The accessory device can include one or more of a suction catheter, a bronchoscope, and a drug delivery catheter. The second conduit can further include a second connection member configured to couple to the housing and allow rotational movement of the second conduit relative to the housing. The forth port can include a sealing member that prevents fluid flow therethrough when the accessory device is uncoupled from the fourth port.

In another interrelated aspect of the current subject matter, a method includes coupling, at a first port of the respiratory valve apparatus, a connecting end of a ventilator. The respiratory valve apparatus can include a second port configured to releasably couple to a resuscitation bag or a transport ventilator, a third port configured to releasably couple to an endotracheal tube, and a piston slidably disposed within a housing of the respiratory valve apparatus. The piston can be biased in a first position within the housing. The piston can include a first flow pathway that allows fluid flow between the first port and the third port when the piston is in the first position and a second flow pathway that allows fluid flow between the second port and the third port when the piston is in a second position. The method can further include coupling, at the third port of the respiratory valve apparatus, a connecting end of the endotracheal tube. Additionally, the method can include coupling, at the second port of the respiratory valve apparatus, a connecting end of the resuscitation bag or the transport ventilator. Furthermore, the coupling of the resuscitation bag or the transport ventilator to the second port forces the piston into a second position can thereby allow fluid flow between the second port and the third port and prevent fluid flow between the first port and the third port.

In some embodiments, the method can include uncoupling the connecting end of the resuscitation bag or the transport ventilator from the second port thereby allowing the piston to move into the first position to allow fluid flow between the first port and the third port and prevent fluid flow between the second port and the third port.

In some embodiments, the respiratory valve apparatus can include a second conduit including the third port and a forth port configured to releasably couple an accessory device. In some embodiments, the method can further include coupling one or more of a suction catheter, a bronchoscope, and a drug delivery catheter to the third port and/or the fourth port.

In another interrelated aspect, a method includes coupling, at a first port of the respiratory valve apparatus, a connecting end of a ventilator. The respiratory valve apparatus can include a second port configured to releasably couple to a resuscitation bag or a transport ventilator, a third port configured to releasably couple to an endotracheal tube, and a lever member pivotably disposed within a flow pathway extending along a housing of the respiratory valve apparatus. The lever member being biased in a first position to allow fluid flow between the first port and the third port. The method can further include coupling, at the third port of the respiratory valve apparatus, a connecting end of the endotracheal tube. The method can further include coupling, at the second port of the respiratory valve apparatus, a connecting end of the resuscitation bag or the transport ventilator. Furthermore, coupling of the resuscitation bag or the transport ventilator to the second port can force the lever member to pivot into a second position thereby allowing fluid flow between the second port and the third port and preventing fluid flow between the first port and the third port.

In some embodiments, the method can include uncoupling the connecting end of the resuscitation bag or the transport ventilator from the second port thereby allowing the lever member to pivot into the first position to allow fluid flow between the first port and the third port and prevent fluid flow between the second port and the third port.

In some embodiments, the respiratory valve apparatus can include a second conduit including the third port and a forth port configured to releasably couple an accessory device. In some embodiments, the method can further include coupling one or more of a suction catheter, a bronchoscope, and a drug delivery catheter to the third port and/or the fourth port.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 shows a perspective side view of an embodiment of a respiratory valve apparatus described herein;

FIG. 2 shows a cross-section of the respiratory valve apparatus in a first configuration including a piston in a first position thereby creating a first air pathway between a patient and a ventilator;

FIG. 3 shows a cross section of the respiratory valve apparatus in a second configuration including the piston in a second position thereby creating a second air pathway between the patient and a manual resuscitator or Ambu bag;

FIG. 4 shows a perspective side view of the piston according to an embodiment of the disclosure;

FIG. 5 shows a top-down view of the housing the respiratory valve apparatus including the resuscitation bag port having the piston therein;

FIG. 6 shows a perspective side view of the piston according to another embodiment of the disclosure;

FIG. 7 shows a cross-section of the respiratory valve apparatus according to another embodiment of the disclosure in a first configuration including the piston of FIG. 6 in a first position thereby creating a first air pathway between a patient and a ventilator;

FIG. 8 shows a cross-section of the respiratory valve apparatus according to another embodiment of the disclosure in a second configuration including the piston of FIG. 6 in a second position thereby creating a second air pathway between the patient and a manual resuscitator or Ambu bag and/or a transport ventilator;

FIG. 9 shows a perspective side view of the piston according to another embodiment of the disclosure;

FIG. 10 shows an enlarged cross-section of a portion of the respiratory valve apparatus according to another embodiment of the disclosure in a first configuration including a lever member thereby creating a first air pathway between the patient and a ventilator;

FIG. 11 shows an enlarged cross-section of a portion of the respiratory valve apparatus according to another embodiment of the disclosure in a second configuration including a lever member thereby creating a second air pathway between the patient and the manual resuscitator or Ambu bag;

FIG. 12 shows an embodiment of a respiratory valve apparatus;

FIG. 13 shows a perspective side view of an embodiment of a respiratory valve apparatus;

FIGS. 14-15 show a side view of an embodiment of a respiratory valve apparatus;

FIGS. 16A-16C show side views of embodiments of a respiratory valve apparatus coupled to a closed suction catheter system;

FIG. 16D shows a side perspective view of an embodiment of a respiratory valve apparatus with a fluid line connected to an accessory port;

FIG. 16E shows a perspective view of an embodiment of a respiratory valve apparatus in use with a patient;

FIG. 17 shows an embodiment of a respiratory valve apparatus including a one-way valve;

FIG. 18 shows an embodiment of a respiratory valve apparatus with a procedure module detached from the airflow module;

FIG. 19 shows a perspective side view of an embodiment of a respiratory valve apparatus connected to a ventilator circuit;

FIG. 20 shows a perspective side view of an embodiment of a respiratory valve apparatus with a closed suction catheter coupled to the accessory port;

FIG. 21 shows a perspective side view of an embodiment of a respiratory valve apparatus with a procedure module detached from an airflow module;

FIGS. 22A and 22B show perspective side views of an embodiment of a respiratory valve apparatus with an airflow module connected to a closed suction catheter;

FIG. 23 shows a perspective side view of an embodiment of a respiratory valve apparatus with a resuscitation bag inserted;

FIG. 24 shows a perspective side view of an embodiment of a respiratory valve apparatus including a transport ventilator connector and/or transport ventilator tube;

FIG. 25 shows a method diagram for connecting a respiratory valve apparatus to a ventilator circuit with a closed suction catheter inserted;

FIG. 26 shows a method diagram for connecting a respiratory valve apparatus to a closed suction catheter;

FIG. 27 shows a method diagram for changing a ventilation source of a respiratory valve apparatus to a resuscitation bag; and

FIG. 28 shows a method diagram for changing a ventilation source of a respiratory valve apparatus to a transport ventilator.

When practical, similar reference numbers denote similar structures, features, or elements.

### DETAILED DESCRIPTION

Implementations of the current subject matter include methods, apparatuses, articles of manufacture, and systems relating to various embodiments of a respiratory valve apparatus, including a modular respiratory valve apparatus that can releasably couple to a variety of devices (e.g., ventilator, endotracheal tube, etc.) in a variety of different configurations. Additionally, the respiratory valve apparatus embodiments described herein can include features for assisting with maintaining a closed ventilator circuit (e.g., exhaled air from patient is at least substantially prevented from being released from respiratory valve apparatus), such as during coupling and uncoupling of devices relative to the respiratory valve apparatus. Such closed circuit ventilator provided by the respiratory valve apparatus can reduce or eliminate harmful exposure between patient and caregiver, as well as reduce or eliminate contamination of the respiratory valve apparatus and/or devices coupled to the respiratory valve apparatus.

As described herein, a ventilator circuit can include an airflow pathway of the respiratory valve apparatus in fluid communication with a patient. As such, a closed ventilator circuit can include the airflow pathway in communication with the patient (e.g., patient's lungs) and/or with one or more devices coupled to the respiratory valve apparatus. The closed ventilator circuit can limit or prevents flow from escaping the respiratory valve apparatus and, instead, provide a substantially sealed fluid pathway (including airflow pathway) between one or more of a variety of devices and the patient.

In some embodiments, the respiratory valve apparatus includes at least four ports (e.g., a ventilator connection port, a suction catheter port, an endotracheal tube port, and a resuscitation bag port) that enable a patient to be switched from mechanical ventilation (e.g., ventilator) to manual ventilation (e.g., using a resuscitation bag and/or transport ventilator) without the need to open ventilator circuit. As such, switching between mechanical and manual ventilation using the respiratory valve apparatus can substantially prevent air entering or exiting the respiratory valve apparatus. This can reduce the risk of ventilator-acquired pneumonia and loss of positive-end expiratory pressure (PEEP) for the patient. The risk of the healthcare worker being exposed to any fluids contained within the ventilator system is also reduced or eliminated with the respiratory valve apparatus described herein.

Patients may have fluid drawn from their lungs as often as six times a day and sometimes more, possibly over long periods of time. As such, the respiratory valve apparatus can minimize patient discomfort while allowing various procedures (e.g., bronchoscopies) and pulmonary function tests to be performed, such as before weaning a patient off a ventilator. In addition, such a device can be widely used in treating pediatric patients, especially premature infants, as well as adults, who are subject to respiratory problems and may need frequent aspirations. As such, it is desirable to simplify such devices and reduce the number of parts in order to reduce costs and increase reliability.

The respiratory valve apparatus described herein provides improvements over other valves, including other respiratory valves. For example, the respiratory valve apparatus disclosed herein can be modular (e.g., form various configurations and/or can be separated into more than one parts) and can include a moveable piston that allows for at least two alternate air pathways as a result of movement by the piston. In addition, the respiratory valve apparatus described herein provides improved transition between mechanical and manual ventilation and/or to a transport ventilator. Furthermore, the respiratory valve apparatus described herein provides reduced dead space within the valve. Such improvements (either alone or in combination) can assist with maintaining positive-end pressure in the patient and reduce the risk of the patient rebreathing CO₂ during ventilation. Other features and benefits of the respiratory valve apparatus are described herein and/or are within the scope of this disclosure.

In some embodiments, the respiratory valve apparatus can include at least one or more of the following features: at least four main ports (e.g., a ventilator connection port, a suction catheter port, an endotracheal tube port, and a resuscitation bag port); the ability to switch from mechanical ventilation to manual ventilation (e.g., via a resuscitation bag) without opening of the ventilation circuit and/or transport ventilator; the ability to conduct pulmonary function tests without opening the ventilator circuit; a piston mechanism within the respiratory valve apparatus that securely switches ventilation control between a ventilator to a resuscitation bag (e.g., as the resuscitation bag is placed on or in connection with the resuscitation bag port); a suction catheter port that is directly in-line with the patient and has a sealing member to allow for medical procedures (e.g., bronchoscopy, drug delivery) to be carried out without interference from ventilator airflow and minimal risk of contamination; an irrigation port positioned along the suction catheter port; and swivel connectors along the respiratory valve apparatus to allow for ergonomic positioning of the respiratory valve apparatus regardless of which ports are in use.

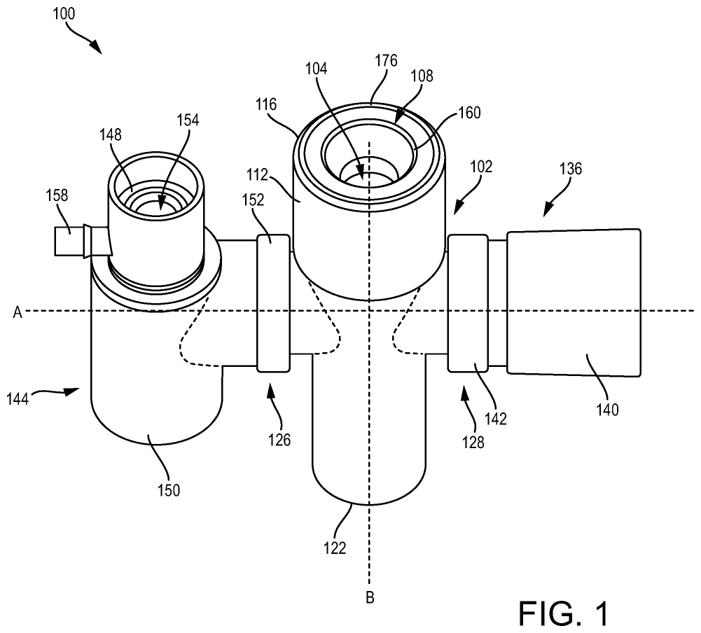

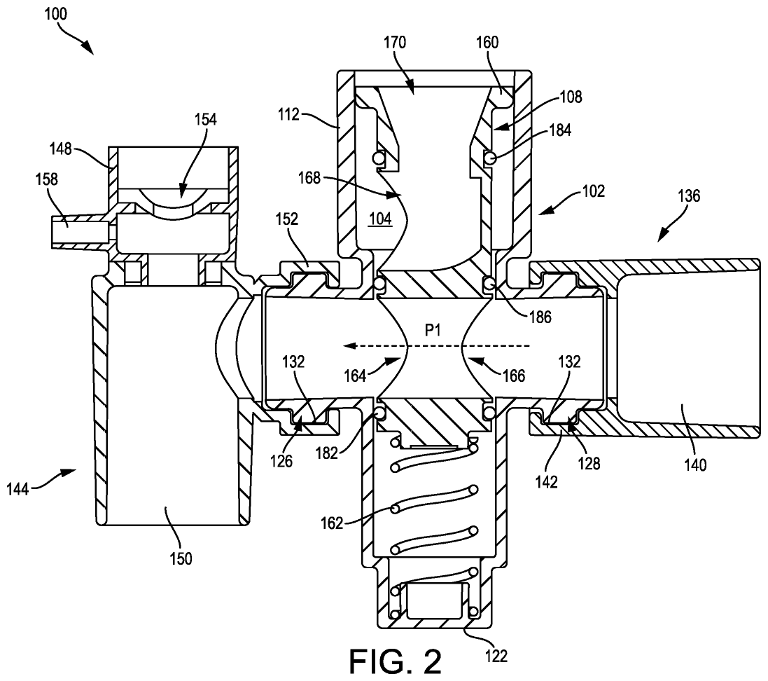

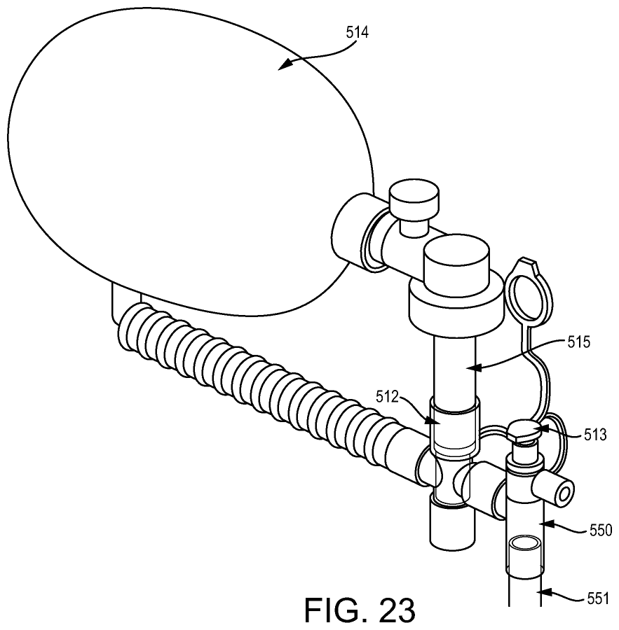

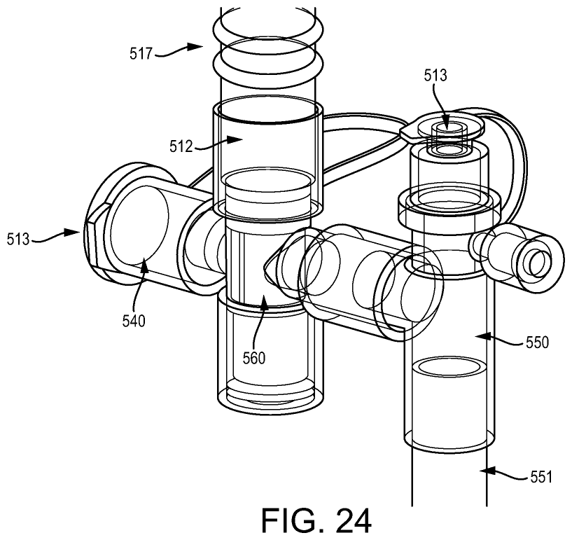

Turning now to FIGS. 1-2, an embodiment of a respiratory valve apparatus 100 is shown. Respiratory valve apparatus 100 may include a housing 102 having an inner chamber 104 disposed therein. Housing 102 may be substantially t-shaped or x-shaped and may house a piston assembly 108 within inner chamber 104. As will be described herein, piston assembly 108 may control airflow between one or more parts of respiratory valve apparatus 100. Housing 102 may also include a resuscitation bag connection port 112 and/or a transport ventilator connector (e.g., transport ventilator connector 518 shown in FIG. 24) on a first end 116 of housing 102. Resuscitation bag connection port 112 may be sized and shaped to accommodate a resuscitation bag (e.g., resuscitation bag 514 shown in FIG. 23) being engaged, connected, attached, joined, linked and/or fastened thereto. For example, resuscitation bag connection port 112 may be threaded or include rotation locking grooves for receiving and retaining the resuscitation bag. However, any conventional means may be used for receiving and retaining the resuscitation bag to the resuscitation bag connection port 112. As described herein, a resuscitation bag can include a bag valve mask, manual resuscitator, a transport ventilator, and/or an Ambu bag, including various embodiments known in the art for using with assisting with respiration.

Housing 102 may include a closed, second end 122 opposite or substantially aligned with first end 116. As will be described herein, second end 122 may function as a spring seat or spring stop when a resuscitation bag is retained on or within resuscitation bag connection port 112. Housing 102 may also include a third end 126 opposing a fourth end 128 along line A, and line A can be normal to a line B defined by first end 116 and second end 122. Each of third end 126 and fourth end 128 may include a connection member 132. Connection members 132 may include, for example, a flange, rib, rim, void, swivel connector, female connection member, and/or a male connection member for securing and/or retaining conduits thereto.

For example, respiratory valve apparatus 100 may also include a conduit 136 including a ventilator connection port 140 and a connection member 142. Connection member 142 may be disposed on an opposing end of conduit 136 from ventilator connection port 140. Connection member 142 may include any means for coupling with connection member 132 of one of third end 126 or fourth end 128 of housing 102 such as, for example, a flange, rib, rim, void, a swivel connector, a female connection member, and/or a male connection member. For example, where connection member 132 includes a male connection member, connection member 142 may include a female connection member. Connection members 132, 142 may couple, connect, attach, join, link and/or fasten such that conduit 136 is in fluid communication with housing 102. While conduit 136 is shown as being coupled with fourth end 128 of housing 102, it is to be understood that conduit 136 may instead couple with third end 126. Connection members 132, 142 may allow for ergonomic positioning of conduit 136 relative to housing 102 thereby aiding in the use of ventilator connection port 140 and resuscitation bag connection port 112.

For example, connection members 132, 142 may engage such that connection members 132, 142 allow 360° rotation of conduit 136 to prevent kinking, twisting, or tangling of the artificial airway circuit. Ventilator connection port 140 may also be known as a respirator connection port, and may be sized and shaped to accommodate a respirator or ventilator (not shown) being engaged, connected, attached, joined, linked and/or fastened thereto. Ventilator connection port 140 may be threaded or include rotation locking grooves (not shown) for receiving and retaining the respirator or ventilator. However, any conventional means may be used for receiving and retaining the ventilator within or on ventilator connection port 140.

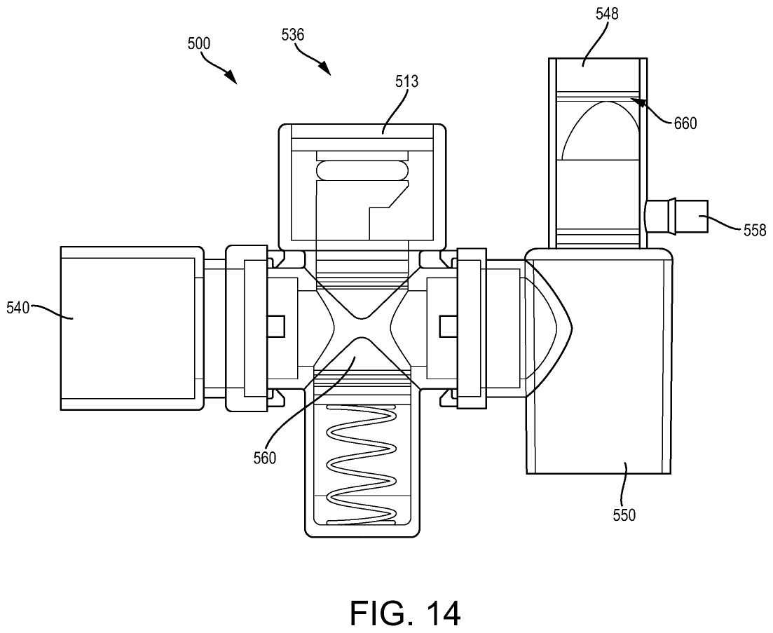

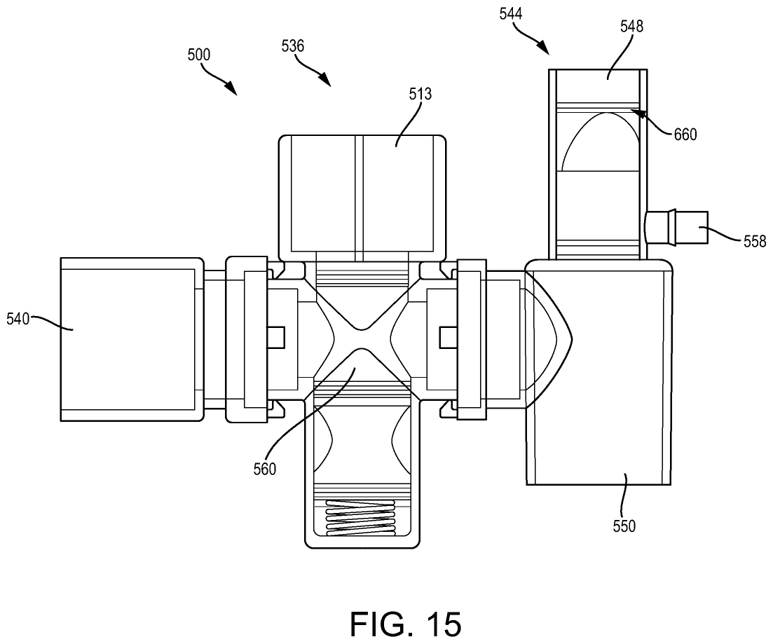

Respiratory valve apparatus 100 may also include a conduit 144 including an accessory port 148, an endotracheal tube connection port 150, and a connection member 152. Accessory port 148 may be sized and shaped for receiving any desirable medical accessory, e.g., a suction catheter, bronchoscope, drug delivery catheter, etc. For example, accessory port 148 may be threaded or include rotation locking grooves for receiving and retaining the accessory. In addition, accessory port 148 may include a sealing member 154 therein. Sealing member 154 may include any means for maintaining a closed ventilator circuit within respiratory valve apparatus 100 (e.g., exhaled air from patient prevented from entering surrounding atmosphere) when accessory port 148 is or is not in use, i.e., when an accessory such as a suction catheter, bronchoscope, or drug delivery catheter is or is not being used. For example, sealing member 154 may include, for example, a dome valve, a duck-billed valve (e.g., duck-billed valve 660 in FIGS. 14-15), a septum, O-ring, or combinations thereof. In some embodiments, sealing member 154 may include a flexible orifice or resealable entry. In some embodiments, sealing member 154 may be closed with a cap of resilient material having diametrical cuts forming openable flaps. Any one or more of the ports of the respiratory valve apparatus described herein can include sealing member 154 for assisting with maintaining a closed ventilator circuit.

In some embodiments, endotracheal tube connection port 150 (e.g., a patient port) may be sized and shaped for receiving an endotracheal tube being coupled thereto. For example, endotracheal tube connection port 150 may be threaded or include rotation locking grooves for receiving and retaining the endotracheal tube. However, any conventional means may be used for receiving and retaining the endotracheal tube within or on endotracheal tube connection port 150. The endotracheal tube may be at least partially disposed within a patient, e.g., within a trachea of the patient.

Connection member 152 may include any means for coupling with connection members 132 one of third end 126 or fourth end 128 of housing 102 opposite of conduit 136. Connection member 152 may include, for example, a flange, rib, rim, void, swivel connector, female connection member, and/or a male connection member. For example, where connection member 132 includes a male connection member, connection member 152 may include a female connection member. Connection members 152, 142 may couple, connect, attach, join, link and/or fasten such that conduit 144 is in fluid communication with housing 102. While conduit 144 is shown as being coupled with third end 126 of housing 102, it is to be understood that conduit 144 may instead couple with fourth end 128. Connection members 152, 142 may allow for ergonomic positioning of conduit 144 relative to housing 102 thereby aiding in the use of accessory port 148 and endotracheal tube connection port 150. For example, connection members 132, 152 may engage such that connection members 132, 152 allow 360° rotation of conduit 144 to prevent kinking, twisting, or tangling of the artificial airway circuit.

As shown in FIGS. 1-2, conduit 144 may be substantially T-shaped such that accessory port 148 may be substantially aligned with and in fluid communication with endotracheal tube connection port 150. That is, accessory port 148 may be directly in-line with endotracheal tube connection port 150. In another embodiment, conduit 144 may be substantially L-shaped such that endotracheal tube connection port 150 and ventilator connection port 140 are in substantial alignment.

In some embodiments, respiratory valve apparatus 100 may also include another accessory port 158. Accessory port 158 may be optional and can include, e.g., an injection port or saline port when, for example, a suction catheter is used in connection with accessory port 148. Accessory port 158 may be sized and shaped for receiving another accessory such as a saline tube being engaged, connected, attached, joined, linked and/or fastened thereto.

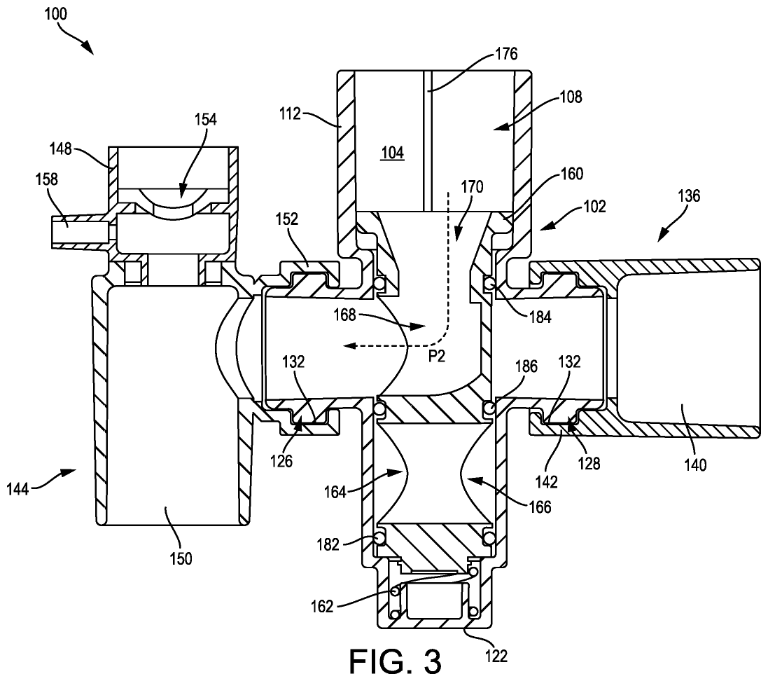

Referring now to FIG. 2-3, piston assembly 108 may include a piston 160 and a spring 162. As described herein, piston assembly 108 can allow two alternate air pathways, such as two alternate pathways with at least one pathway passing through piston 160. In addition to other benefits, piston assembly 108 minimizes dead space within respiratory valve apparatus 100, which in turn helps to maintain positive-end pressure, reduces the risk of the patient rebreathing CO₂ during ventilation, and reduces moisture buildup within respiratory valve apparatus 100. Piston assembly 108 may be disposed within inner chamber 104 of housing 102 and extend substantially along line B (FIG. 1). That is, piston assembly 108 may be disposed within housing 102 such that piston assembly 108 extends within inner chamber 104 between first end 116 having resuscitation bag connection port 112 and closed, second end 122. As shown in FIG. 2-3, spring 162 may be disposed between piston 160 and second end 122 of housing 102, while piston 160 may extend at least partially within resuscitation bag connection port 112 when spring 162 is in a non-compressed state. Piston assembly 108 or more specifically, piston 160 may be substantially cylindrical in shape and may include two distinct airflow passageways therein. Any number of biasing elements, including various embodiments of the spring 162, are within the scope of this disclosure for biasing and assisting with moving the piston. Furthermore, the biasing element, including the spring 162, can be made out of one or more of a variety of materials, including various metals and plastics.

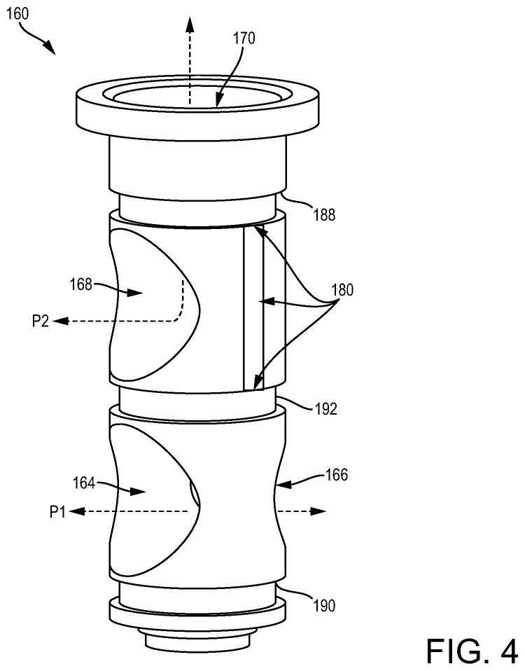

FIG. 4 shows an enlarged perspective view of an embodiment of the piston 160. Referring now to FIG. 4 together with FIGS. 2-3, piston 160 may include a first opening 164 and a second opening 166 defining a first flow pathway or passageway (designated by dotted line arrows P1 (FIG. 2)). First passageway P1 may provide a first flow pathway between endotracheal tube connection port 150 (FIGS. 2-3) and ventilator connection port 140 (FIGS. 2-3) when piston 160 is in a first position, or when spring 162 is in a non-compressed state (FIG. 2). First passageway P1 may extend normal to a longitudinal axis of piston 160. Piston 160 may include a third opening 168 and a fourth opening 170 defining a second flow pathway or passageway (designated by dotted line arrows P2 (FIG. 3)). Second passageway P2 may extend both normal and parallel to the longitudinal axis of piston 160. Second passageway P2 may provide a second flow pathway between endotracheal tube connection port 150 and resuscitation bag connection port 112 (FIGS. 2-3) when in a second position, or when spring 162 is compressed (FIG. 3).

Referring only now to FIGS. 2-3, spring 162 may include, e.g., a compression spring. Spring 162 is to be used to securely move piston 160 thereby controlling the airflow from a ventilator (not shown) positioned at or within ventilator connection port 140 to the resuscitation bag positioned at or within resuscitation bag connection port 112, such as when the resuscitation bag is coupled to respiratory valve apparatus 100. Spring 162 may bias the piston 160 in the first position thereby defining first flow pathway P1. Spring 162 may be attached to, e.g., via an adhesive to an end of piston 160 that is disposed furthest from resuscitation bag connection port 112. However, spring 162 may be attached to piston 160 via any other reasonable means without departing from aspects of the disclosure, and may even merely rest against or contact the piston 160 without being formally attached thereto.

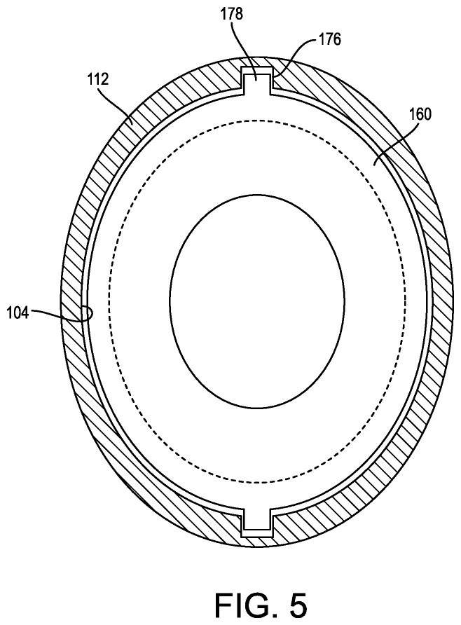

Piston 160 may be retained within optional rotational locking grooves 176 (FIGS. 1 and 5), e.g., at least two grooves. Rotational locking grooves 176 may be formed within internal walls of housing 102. Rotational locking grooves 176 may couple with locking tabs 178 (FIG. 5) on an external wall of at least a portion of piston 160, e.g., a portion of piston 160 that is at least partially disposed within resuscitation bag connection port 112 when respiratory valve apparatus 100 is in the first position (FIG. 2). Piston 160 can be moved within rotational locking grooves 176 between positions that affect the flow of air, i.e., up and down on the pages including FIGS. 2 and 3. Rotational locking grooves 176 may prevent piston 160 from rotating about the longitudinal axis of piston 160 within housing 102. This ensures proper alignment of flow pathways P1, P2 through piston 160 to line up with chambers in housing 102.

First and second flow pathways P1, P2 provided by piston 160 can be sealed in some embodiments. That is respiratory valve apparatus 100 can include one or more optional seals as may be necessary. For example, respiratory valve apparatus 100 can include a first seal 182 at a first end of piston 160, a second seal 184 at a second end of piston 160, and a third seal 186 positioned along the length of piston 160 between first and second ends of piston 160. More specifically, first seal 182 may be disposed at an end of piston 160 nearest the spring 162 (FIGS. 2-3) or second end 122 of housing 102. Second seal 184 may be disposed at an end of piston 160 furthest from spring 162 and adjacent first end 116 of housing 102. Further, third seal 186 may be disposed at a position between openings 164, 166 defining first passageway P1 (FIG. 2) and openings 168, 170 defining second passageway P2 (FIG. 3) so that passageways P1, P2 are sealed off from one another. Additionally, first seal 182, second seal 184, and third seal 186 can be configured to assist with directing fluid or airflow through housing 102. Furthermore, additional seals can be included in the respiratory valve apparatus 100, such as for providing air and fluid seals along any of the joints in the respiratory valve apparatus 100. For example, additional seals can be included adjacent any of the connection members 142, 152 such as to ensure fluid and/or air seals along respective couplings between the conduits 136, 144 and the housing 110. The seals can be made out of one or more materials, such as various rubber and/or silicone materials, without departing from the scope of this disclosure.

In some embodiments, the first seal 182 and third seal 186 can assist with directing fluid or airflow through first flow pathway P1. Second seal 184 and third seal 186 can assist with directing fluid or airflow through second flow pathway P2. Each of first seal 182, second seal 184, and third seal 186 can include any type of seal for maintaining a closed circuit (e.g., respiratory valve apparatus 100 does not include fluid/air leaks) and assisting with directing fluid or airflow within pathways P1, P2. For example, seals 182, 184, 186 (as well as any other seals of the respiratory valve apparatus 100) may include an O-ring, U-ring, V-ring, lip, double lip, cord ring, piston seal, rod seal, flange, chevron, wiper, etc. In some embodiments, piston 160 may use a custom silicone connected O-ring overmolded and connected by two silicone channels 180 along the piston 160. Seals 182, 184, 186 may be optionally positioned within seal grooves 188, 190, 192 (FIG. 4) within piston 160. Seal grooves 188, 190, 192 may be formed in and extend at least partially around an external surface of piston 160. Seal grooves 188, 190, 192 may be sized and shaped to house the seals 182, 184, 186 such that passageways P1, P2 (FIGS. 2-3) are substantially sealed off from one another. While three seals and three seal grooves are shown, it is to be understood that any number of seals or seal grooves can be included without departing from aspects of this disclosure.

FIGS. 2-3 show the respiratory valve apparatus 100 including piston assembly 108 in two positions during use. FIG. 2 shows respiratory valve apparatus 100 when piston assembly 108 is in a first position, i.e., when no resuscitation bag is placed into/onto the resuscitation bag connection port 112. As shown in FIG. 2, resuscitation bag connection port 112 is closed when piston 160 is in the first position thereby preventing flow through resuscitation bag connection port 112. Spring 162 can bias the piston 160 in the first position thereby creating the first flow pathway P1 to ventilator connection port 140. FIG. 3 shows respiratory valve apparatus 100 when piston assembly 108 is in a second position, i.e., when a resuscitation bag is placed into/onto the resuscitation bag connection port 112. As shown in FIG. 3, ventilator connection port 140 is closed when piston 160 is in the second position thereby preventing flow through ventilator connection port 140. Compression of spring 162 places the piston 160 in the second position thereby creating the second flow pathway P2 to resuscitation bag connection port 112. Once resuscitation bag is placed into/onto the resuscitation bag connection port 112, piston 160 is actuated and spring 162 is compressed. During actuation of piston 160 (or compression of spring 162), piston 160 is forced in a direction toward second end 122 of housing 102 which acts as a spring seat or stop for spring 162. In some embodiments, internal walls of housing 102 of resuscitation bag connection port 112 can be sloped to provide frictional forces along the wall of resuscitation bag connection port 112 as piston 160 reaches appropriate displacement thereby locking the resuscitation bag, and subsequently the piston 160, in position for flow channels of endotracheal tube connection port 150 and resuscitation bag connection port 112 to become in fluid communication with one another. When the resuscitation bag is removed from resuscitation bag connection port 112, piston 160 is returned to a resting position and spring 162 is expanded. Air can then pass through the ventilator pathway for mechanical ventilation of the patient (see FIG. 2).

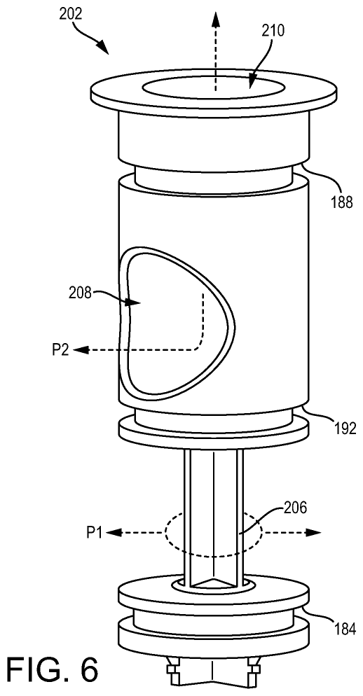

FIG. 6 shows a perspective view of a piston 202 that can be used with respiratory valve apparatus 100 according to another embodiment of the disclosure. Unlike piston 160, piston 202 does not include openings 164, 166. Rather, piston 202 includes a pillar 206 that is sized and shaped to allow air or fluid to pass through piston by passing around pillar 206 thereby defining first passageway P1. Like piston 160, piston 202 includes openings 208, 210 defining second passageway P2 as was described relative to FIGS. 1-2.

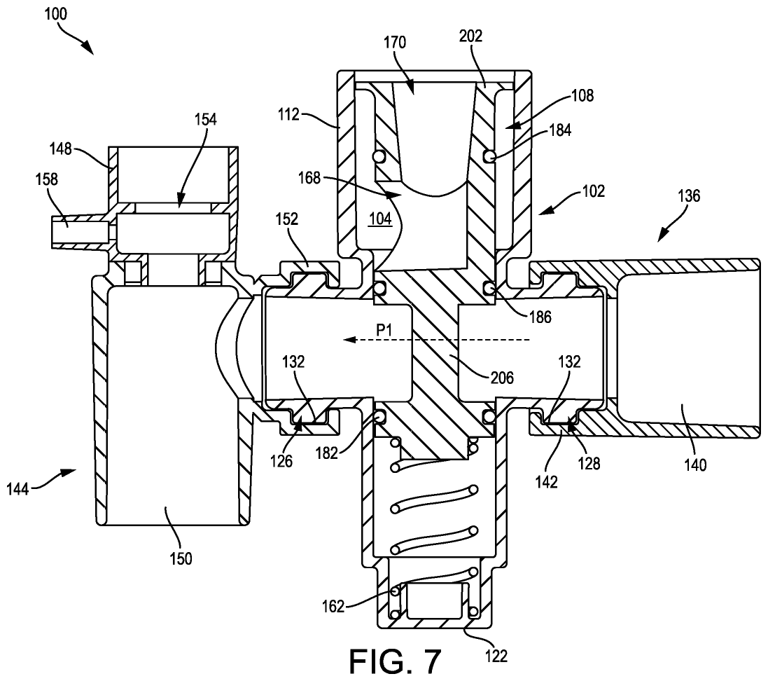

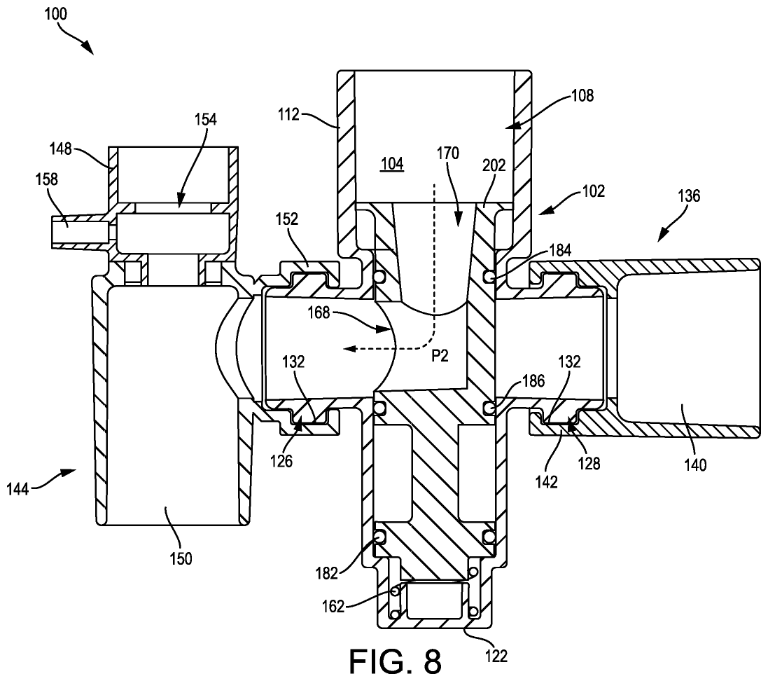

FIGS. 7-8 show the respiratory valve apparatus 100 including piston assembly 108 having piston 202 in two possible positions during use. FIG. 7 shows respiratory valve apparatus 100 when piston assembly 108 is in a first portion, i.e., when no resuscitation bag is placed into/onto the resuscitation bag connection port 112. As shown in FIG. 7, resuscitation bag connection port 112 is closed when piston 202 is in the first position thereby preventing flow through resuscitation bag connection port 112. Spring 162 biases the piston 202 in the first position thereby creating the first flow pathway P1 to ventilator connection port 140. FIG. 8 shows respiratory valve apparatus 100 when piston assembly 108 is in a second position, i.e., when a resuscitation bag is placed into/onto the resuscitation bag connection port 112, thereby pushing piston 202 into second position. As shown in FIG. 8, ventilator connection port 140 is closed when piston 202 is in the second position thereby preventing flow through ventilator connection port 140. Compression of spring 162 places the piston 202 in the second position thereby creating the second flow pathway P2 to resuscitation bag connection port 112.

For example, once the resuscitation bag is placed into/onto the resuscitation bag connection port 112, piston 202 is actuated and spring 162 is compressed. During actuation of piston 202 (or compression of spring 162), piston 202 is forced in a direction toward closed, second end 122 of housing 102 which acts as a spring seat or stop for spring 162. In some embodiments, internal walls of housing 102 of resuscitation bag connection port 112 can be sloped to provide frictional forces along the wall of resuscitation bag connection port 112 as piston 202 reaches appropriate displacement thereby locking the resuscitation bag, and subsequently the piston 202, in position for flow channels of endotracheal tube connection port 150 and resuscitation bag connection port 112 to become in fluid communication with one another. When the resuscitation bag is removed from resuscitation bag connection port 112, piston 202 is returned to a resting position (as shown in FIG. 7) as spring 162 is expanded. Air or fluid can then pass through respiratory valve apparatus 100 between ventilator connection port 140 and endotracheal tube connection port 150 for mechanical ventilation of the patient (see FIG. 8).

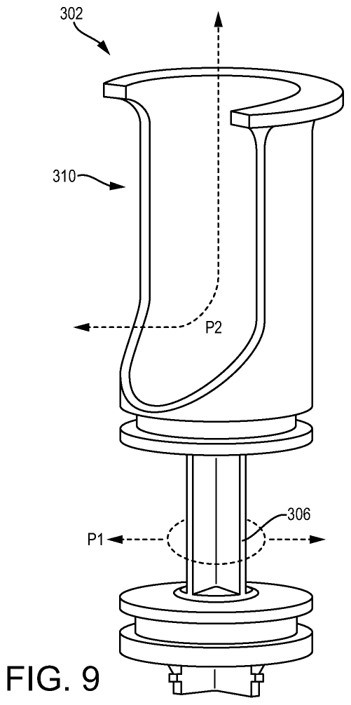

FIG. 9 shows a perspective view of a piston 302 that can be used with respiratory valve apparatus 100 according to another embodiment of the disclosure. As shown in FIG. 9, piston 302 includes a pillar 306 that is sized and shaped to allow air or fluid to pass through piston 302 by passing around pillar 306 thereby defining first passageway P1. Additionally, a portion of the sidewall of the piston 302 at an end nearest the resuscitation bag connection port 112 is removed and is open continuously through to a surface of piston 302 nearest the resuscitation bag connection port 112 (or an upper surface as shown on the page containing FIG. 9). That is, an opening 310 may be disposed through a sidewall of piston 302.

Similar to piston 160 and piston 202, piston 302 may be actuated by use or connection of a resuscitation bag onto/into the resuscitation bag connection port 112 (FIGS. 1-3 and 7-8) and compression of spring 162 (FIGS. 2-3 and 7-8) such that piston 302 can alternate flow through passageway P1 and passageway P2.

Respiratory valve apparatus 100 described herein can provide at least the following: improved transition between mechanical ventilation and manual ventilation; reduced dead space volume within the piston thereby reducing both the risk of losing positive-end pressure within the lungs of the patient and reducing the risk of contamination within the piston (e.g., reduction of infection risk for patient); improved ability to conduct ventilator weaning studies on the patient without risking opening the circuit and removing the patient from mechanical ventilation too early in recovery. Respiratory valve apparatus 100 can be substantially T-shaped, X-shaped, Y-shaped, or t-shaped. Respiratory valve apparatus 100 can include or be a part of a kit having a resuscitation bag adapted to securely connect with the resuscitation bag connection port, an endotracheal tube adapted to securely connect with the endotracheal connection port.

Housing 102 and conduits 136, 144 may be composed of any number of a variety of materials, such a biocompatible materials, including those used for respiratory or medical valves. For example, such materials can include or more of a plastic, a metal, a polymer, polypropylene, medical grade silicone, biocompatible material, etc. Housing 102 may be substantially t-shaped, x-shaped, or any other shape that is able to define pathways P1, P2 together with piston assembly 108. Respiratory valve apparatus 100 may be formed by injection molding, rotational molding, blow molding, compression molding, 3D printing or additive manufacturing, machining, etc. Pistons 160, 202, 302 may be composed of one or more of a plastic, a metal, a composite, a polymer, etc. Pistons 160, 202, 302 may be formed by injection molding, rotational molding, blow molding, compression molding, 3D printing or additive manufacturing, machining, etc. The spring 162 (or any biasing member acting against an embodiment of the piston, such as pistons 160, 202, 302) may be composed of one or more of a plastic, a metal, a composite, a polymer, etc.

Additionally, while various embodiments of the piston (e.g., piston 160, 202, 302) have been shown and described herein, other piston embodiments that can allow at least two passageways for fluidly connecting an endotracheal tube port to a ventilator connection port and the endotracheal tube port to a resuscitation bag port (e.g., passageways P1, P2) can be included without departing from the scope of this disclosure. That is, the respiratory valve apparatus 100 can be used together with a piston or feature that is capable of switching from mechanical ventilation to manual ventilation (e.g., via a resuscitation bag) without opening of the ventilation circuit (maintain a closed ventilation circuit). For example, the respiratory valve apparatus 100 can allow the conduction of pulmonary function tests without opening the ventilator circuit, as well as switch from a ventilator to a resuscitation bag as the resuscitation bag is placed on or in connection with the resuscitation bag port without opening the ventilation circuit.

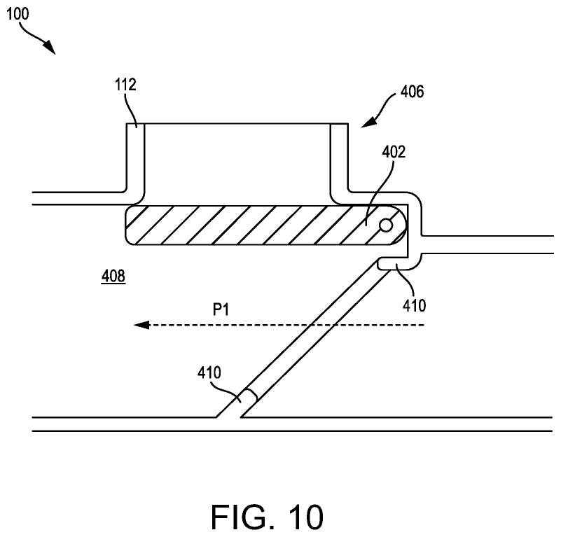

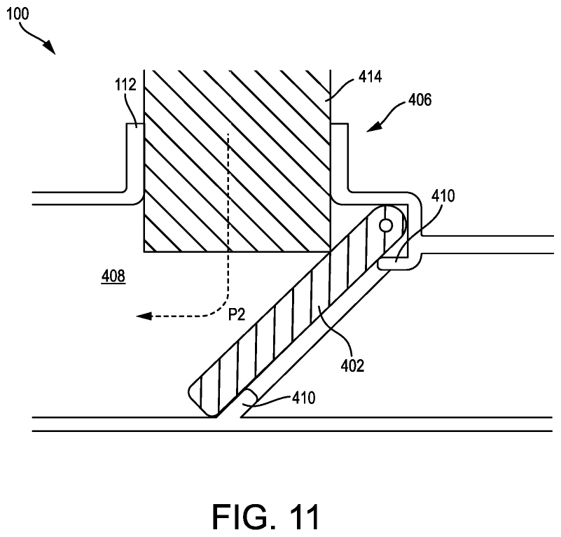

FIGS. 10-11 show an enlarged cross-section of a portion of respiratory valve apparatus 100 according to another embodiment of the disclosure. In this embodiment, the piston of the previous embodiments, e.g., piston 160 (FIGS. 2-4), 202 (FIGS. 6-7), 302 (FIG. 9), is replaced with a lever member 402. FIG. 10 shows respiratory valve apparatus 100 including lever member 402 in a first position defining a first passageway P1 between endotracheal tube connection port 150 and ventilator connection port 140 (as shown in FIGS. 1-3 and 7-8). FIG. 11 shows respiratory valve apparatus 100 including lever member 402 in a second position defining a second passageway P2 between endotracheal tube connection port 150 and resuscitation bag connection port 112.

In this embodiment, respiratory valve apparatus 100 may include housing 406 having an inner chamber 408 therein. Housing 406 may differ from housing 102 (FIGS. 1-3 and 7-8) in that housing 406 is substantially T-shaped and does not require a spring or spring seat. Rather, housing 406 may include at least one projection 410 therein for supporting and positioning the lever member 402. Projection 410 may include, for example, a lip or a rib. Projection 410 may be disposed or positioned within inner chamber 408 such that projection 410 aids the lever member 402 in sealing passageways P1, P2 and may act as or provide a seat or stop for lever member 402. Projection 410 may be integrally formed within housing 406 and/or may be a separate member attached therein. Projection 410 may include a single unitary body that extends circumferentially about inner chamber 408 within housing 406. Projection 410 may be angled about inner chamber 408 at any angle sufficient to aid the lever member 402 with producing a fluidic seal (preventing fluid flow along P1) when in the second position (FIG. 11). In another embodiment, projection 410 may include separate projections circumferentially spaced about inner chamber 408.

Lever member 402 may be pivotably disposed within the inner chamber 408. Lever member 402 may include any type of bar, rod, flap, etc. Lever member 402 may be any shape or type of member for substantially sealing passageways P1, P2. As shown in FIGS. 10-11, lever member 402 may be pivotable from a first position (FIG. 10) to a second position (FIG. 11). That is, lever member 402 may be pivotably attached to housing 406 within inner chamber 408. Lever member 402 may be biased (e.g., spring-loaded) in the first position such that when there is no resuscitation bag disposed within resuscitation bag connection port 112, lever member 402 defines first passageway P1 between endotracheal tube connection port 150 and ventilator connection port 140. Additionally, resuscitation bag connection port 112 is closed when lever member 402 is in the first position thereby preventing flow through resuscitation bag connection port 112. Lever member 402 may be biased, for example, via the material properties of lever member 402, a flat spring, a compression spring, a torsion spring, etc., for biasing the lever member 402 in the first position thereby preventing flow through resuscitation bag connection port 112. The biasing of lever member 402 in the first position may be assisted by the fluid pressure (air pressure) of a ventilator disposed within or on ventilator connection port 140 (FIGS. 1-3 and 7-8).

As shown in FIG. 11, lever member 402 may be pivotable to the second position thereby defining a second passageway P2 between the endotracheal tube connection port 150 and resuscitation bag connection port 112. That is, lever member 402 may be actuated, or may switch to the second position, upon insertion of a connecting end of a resuscitation bag 414 (FIG. 11) within resuscitation bag connection port 112. More specifically, as shown in FIG. 11, upon insertion of resuscitation bag 414, resuscitation bag 414 may contact the lever member 402 and force the lever member 402 in a direction toward projections 410 to pivot the lever member 402 from the first position to the second position such that ventilator connection port 140 is closed and resuscitation bag connection port 112 is open. That is, ventilator connection port is closed when lever member 402 is in the second position thereby preventing flow through ventilator connection port 140. Lever member 402 may be held in place in the second position so long as resuscitation bag 414 remains disposed within resuscitation bag connection port 112. In addition, the fluid pressure (air pressure) of resuscitation bag 414 may provide additional force to lever member 402 to maintain lever member 402 in the second position and aid in sealing second passageway P2 (FIG. 11). Once the resuscitation bag 414 is removed from resuscitation bag connection port 112, lever member 402 may be returned to the first position (FIG. 10). That is, once the resuscitation bag 414 is removed and no longer applies a force to lever member 402, lever member 402 can return to the first position since lever member 402 is biased in first position.

Various methods associated with the respiratory valve apparatus 100 are within the scope of this disclosure. For example, a method may include providing respiratory valve apparatus 100 according to at least one of the embodiments described herein and coupling a connecting end of a resuscitation bag (e.g., resuscitation bag 414 (FIG. 11)) to resuscitation bag connection port 112 thereby actuating the piston assembly 108 (FIGS. 2-4 and 6-9) or lever member 402 (FIGS. 10-11) such that first passageway P1 (i.e., the passageway between endotracheal tube connection port 150 between ventilator connection port 140) closes, and the second passageway P2 (i.e., the passageway between endotracheal tube connection port 150 and resuscitation bag connection port 112) opens.

In some embodiments, the actuating of piston assembly 108 may include compressing the spring 162 disposed within inner chamber 104 of housing 102. Further, once resuscitation bag is no longer needed or in use, the method may include removing the resuscitation bag from resuscitation bag connection port 112 thereby actuating the piston assembly 108 such that first passageway P1 opens and second passageway P2 closes. The actuating of piston assembly 108 such that first passageway P1 opens and second passageway P2 closes may include expanding the spring 162 disposed within inner chamber 104 of housing 102. In another embodiment, the actuating of lever member 402 may include forcing or causing lever member 402 to contact the projection 410. The placing of the resuscitation bag onto resuscitation bag connection port 112 and the removing of the resuscitation bag from resuscitation bag connection port 112 may be performed such that respiratory valve apparatus 100 remains a closed ventilator circuit during operation.

As mentioned above, conventional ventilator circuits generally include tubing, adaptors, and other components configured to direct air from a ventilator to an endotracheal tube and to the patient. Over time during use, mucus may build up in the endotracheal tube which may require a disconnection in the ventilator circuit to expose the endotracheal tube for cleaning, such as with a closed suction catheter. During this disconnection, exhausted breathe from the patient can be released into the ambient atmosphere thereby contaminating the area and creating a greater possibility of infecting caretakers and other patients. By reducing the number of circuit openings and eliminating the need to disconnect the endotracheal tube, the current design provides a safer ventilator circuit, especially when treating exceptionally infectious viruses.

Additionally, in other situations such as patients requiring suctioning, a bronchoscopy, transport, or a resuscitation bag, conventional ventilator circuits may need to be opened to administer such connections, thus risking exposure to others. The current design solves these issues by providing multiple connection ports for various treatments such that the ventilator adapter as described herein maintains a closed ventilator circuit and thus reduces or eliminates the possibility of exposure to infectious agents.

In some embodiments, the respiratory valve apparatus described herein provides improvements over other valves, including other respiratory valves. For example, the respiratory valve apparatus disclosed herein may allow a section of the respiratory valve apparatus including an accessory port and endotracheal tube connection port to be detached. Such detachable features may be useful because the respiratory valve apparatus can be compatible with commercially available suction catheters and suction catheter systems and may be easily integrated in other ventilator circuit configurations.

Described herein includes various embodiments of a respiratory valve apparatus including a ventilator adapter. The ventilator adapter may include an airflow module and a procedure module. The airflow module may include at least three ports (e.g., a ventilator connection port, a resuscitation bag connection port, and an output port), however, more or less ports are within the scope of this disclosure. The procedure module may include at least four ports (e.g., a closed suction catheter port, a saline flush port, an endotracheal tube connection port, and an input port), however, more or less ports are within the scope of this disclosure. The multiple ports of the ventilator adapter, as further described herein, enable health professionals to provide various patient treatments to an intubated patient without the need to open a ventilator circuit.

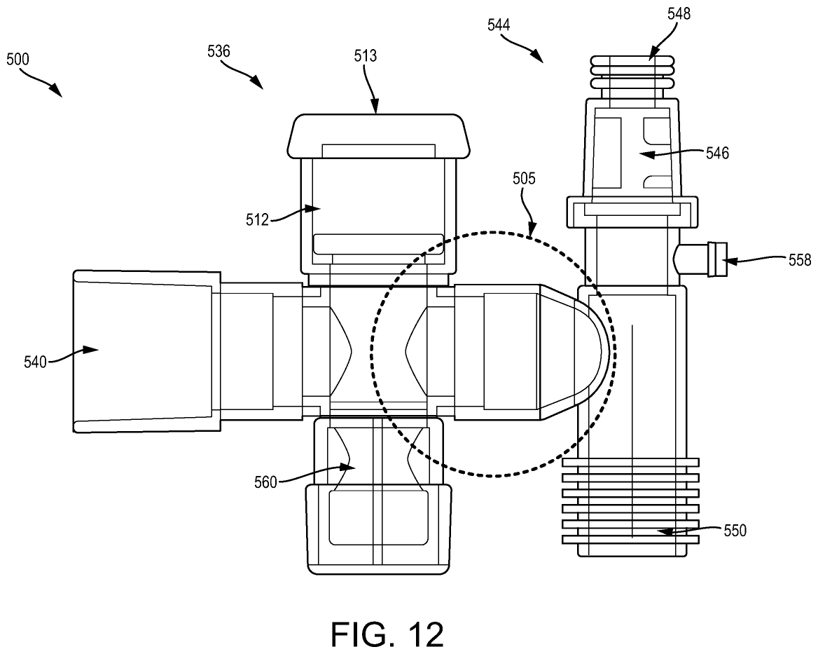

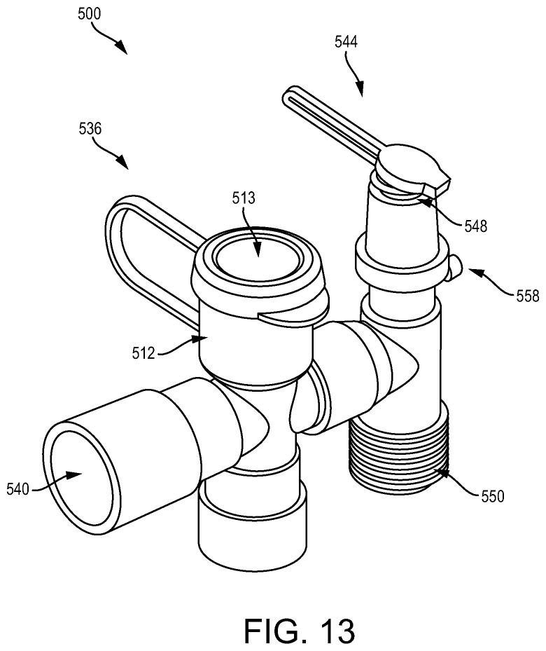

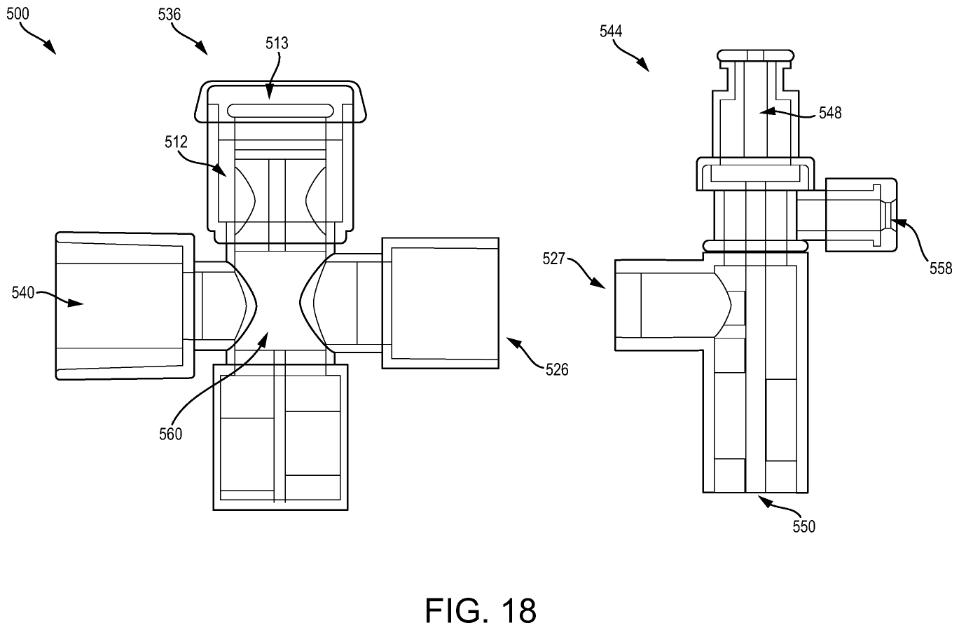

Turning now to FIGS. 12-16, an embodiment of a ventilator adapter 500 is shown. In some embodiments, the ventilator adapter 500 may include an airflow module 536 and a procedure module 544. The airflow module 536 may include a ventilator connection port 540, a resuscitation bag connection port 512, and an output port 526. Resuscitation bag connection port 512 may be sized and shaped to accommodate a resuscitation bag (e.g., resuscitation bag 514 shown in FIG. 23) being engaged, connected, attached, joined, linked and/or fastened thereto. For example, resuscitation bag connection port 512 may be threaded or include rotation locking grooves for receiving and retaining the resuscitation bag. The output port 526 may be positioned opposite or substantially aligned with the ventilator connection port 540, as shown in FIG. 18. The procedure module 544 may include a first accessory port 548, a second accessory port 558, an endotracheal tube connection port 550, and an input port 527. The output port 526 may be configured to couple with the input port 527. In some embodiments, as shown in FIG. 18, the output port 526 may be formed of a shape and/or size different than that of the ventilator connection port 540 such that the ventilator connection port 540 may not be inadvertently coupled with the input port 527. Such a size and/or shape differential may prevent the output port 526 from being inadvertently coupled to the ventilator tubing.

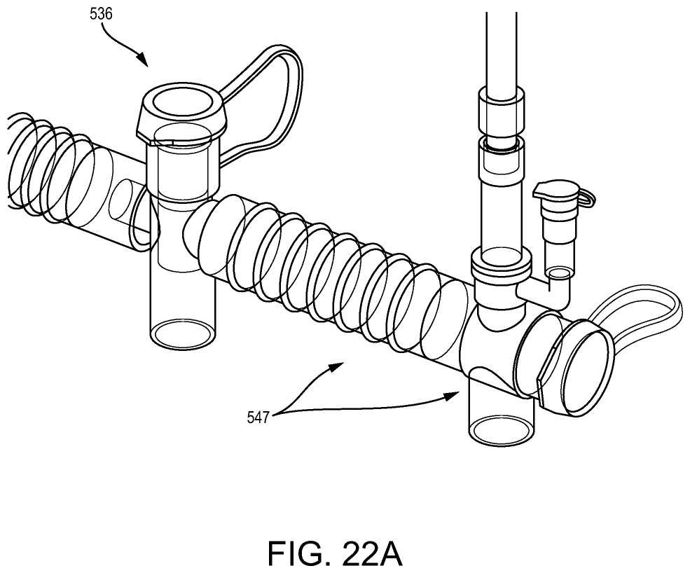

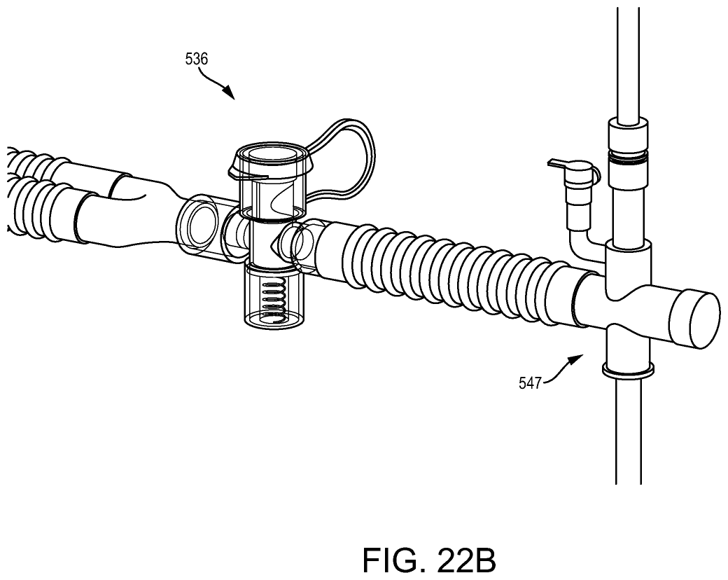

The ventilator adapter 500 may include a detachable double annular connection 505 (shown as coupled in FIG. 12) including an output port 526 and an input port 527 (shown in FIG. 18). The detachable double annular connection 505 may be configured to couple the output port 526 and the input port 527. The detachable double annular connection 505 may include a flange, rib, rim, void, swivel connector, female connection member, and/or a male connection member for securing and/or retaining conduits thereto. For example, the output port 526 may include a swivel feature configured to couple with the input port 527. The output port 526 may include a 22 mm diameter female connection. The input port 527 may include a 15 mm diameter male connection. In some embodiments, the output port 526 and/or input port 527 may be configured to couple to a closed suction catheter or adapter tube, as shown in FIGS. 22A and 22B.

In some embodiments, the respiratory valve apparatus 100 may include a ventilator adapter 500 for use between the ventilator connection port 540 and the endotracheal tube connection port 550. The ventilator adapter 500 may be configured to maintain a closed circuit when transitioning to manual ventilation (e.g., transport or resuscitation), performing in-line suction/flush, or performing bronchoscopy (including up to 16 Fr). The ventilator adapter 500 may be configured to maintain positive end-expiratory pressure (PEEP) and functional residual capacity (FRC) when switching from mechanical to manual ventilation or when performing patient care tasks such as suctioning or bronchoscopy procedures. The ventilator adapter 500 may be configured for single patient use and/or disposable.

The procedure module 544 may include a second accessory port 558, such as a saline flush port, configured to flush saline through the ventilator to clean mucus out through a suction valve. The saline port may include a spring and a silicone tapered bullet. The spring may be configured to apply a force onto the tapered bullet such that the bullet is pushed away from the spring and toward the outer opening of the saline port to seal the saline port opening. A saline tube may be connected to the saline flush port by inserting an end bulb on the saline tube into the outer opening of the saline flush port. For example, as the end bulb of the saline tube is inserted into the saline flush port, the bullet is pushed back and compresses the spring, thus creating a channel configured to allow the saline to flow through the ventilator adapter.

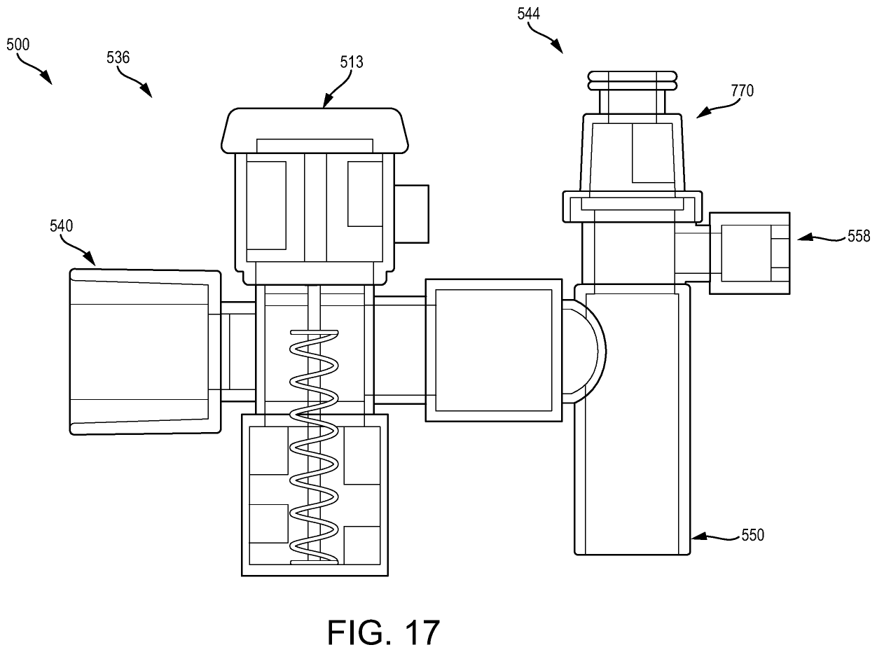

As shown in FIG. 17, in some embodiments, the ventilator adapter 500 may include an airflow module 536 and a procedure module 544. The airflow module 536 may be configured to channel mechanical or manual ventilation to the patient. The procedure module 544 may include a one-way valve 770 configured to provide access for suction catheters and bronchoscopes. In some embodiments, the airflow module 536 may be separated from and/or used without the procedure module 544, as shown in FIG. 18. The ventilator adapter 500 may be configured to mate with at least the following: standard ventilator connection ports, closed suction catheters, closed suction catheter systems, resuscitation bags, bronchoscopes, and/or endotracheal tube systems.

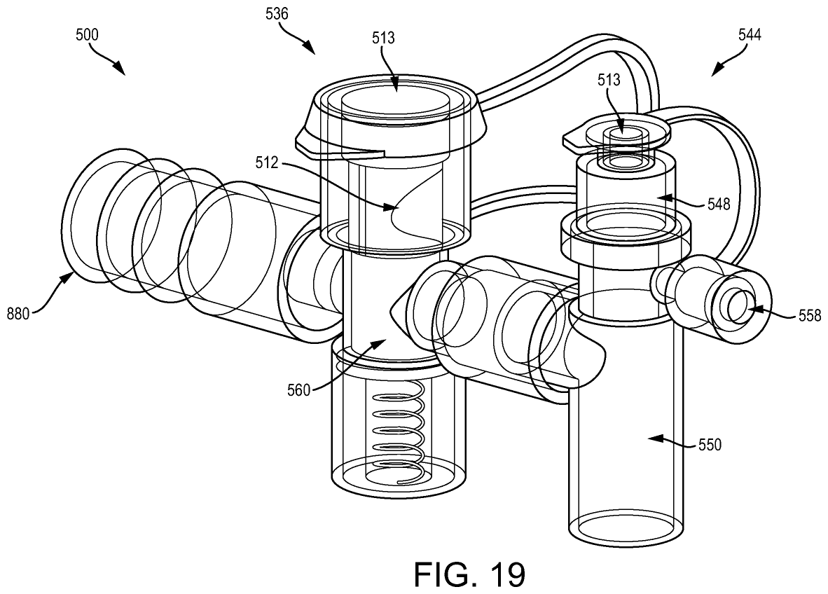

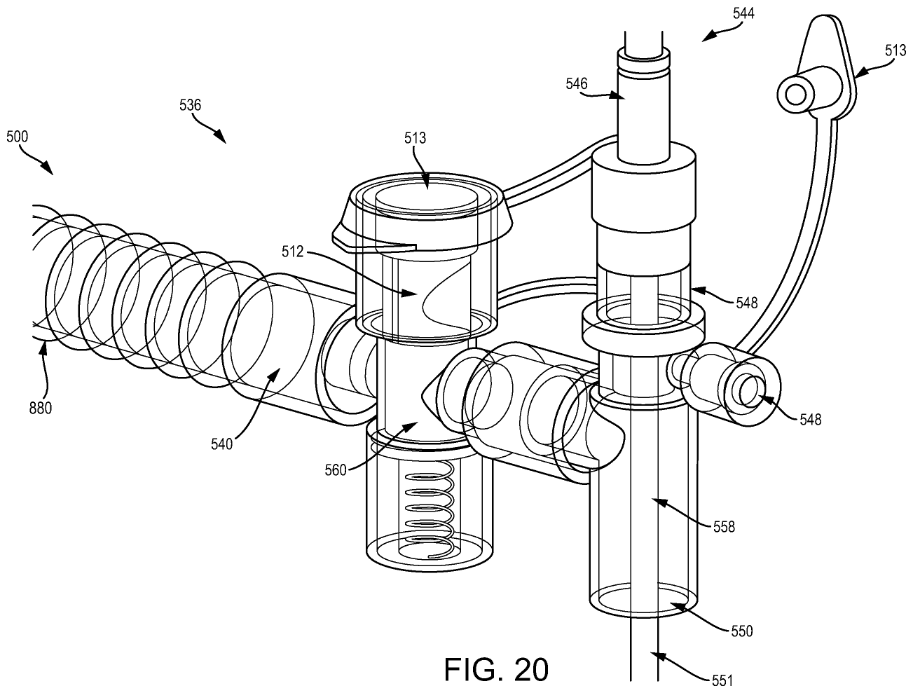

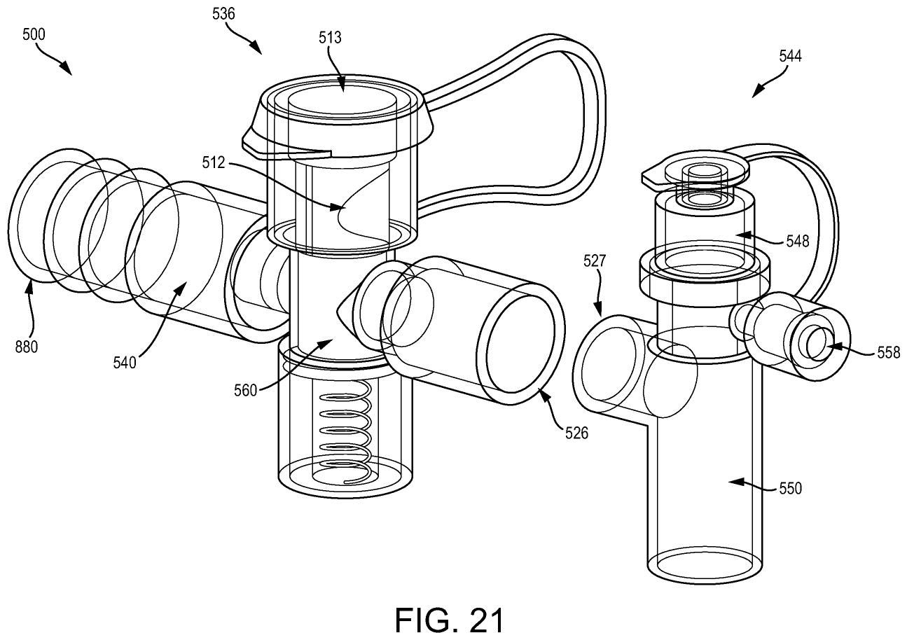

In some embodiments, as shown in FIGS. 19-21, the ventilator adapter 500 may be connected to a ventilator 880 via ventilator connection port 540. For example, a closed suction catheter 546 may be inserted into an accessory port 548, as shown in FIG. 20. The resuscitation bag piston 560 may be positioned in an up or first position, as shown in FIG. 19. The closed suction catheter 546 may be inserted into an accessory port 548 (e.g., after removal of cap 513) of the procedure module 544, as shown in FIG. 20. Optionally, a bronchoscope may be inserted into the accessory port 548 of the procedure module 544 for use with ventilator adapter 500. For example, the ventilator adapter 500 may be configured to accept bronchoscopes up to 16 f. In some embodiments, the ventilator adapter 500 may be connected with a closed suction catheter 546 inserted into the ventilator connection port 540 and/or the endotracheal tube connection port 550.

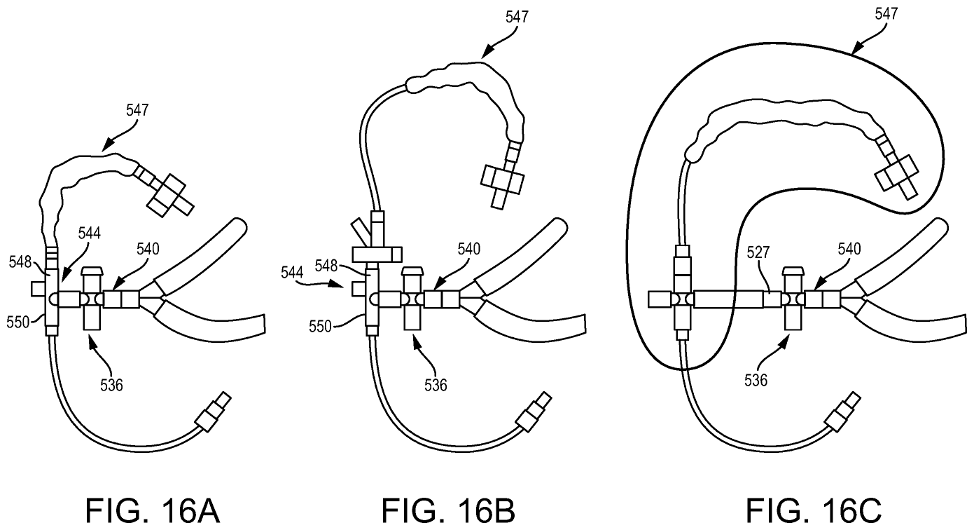

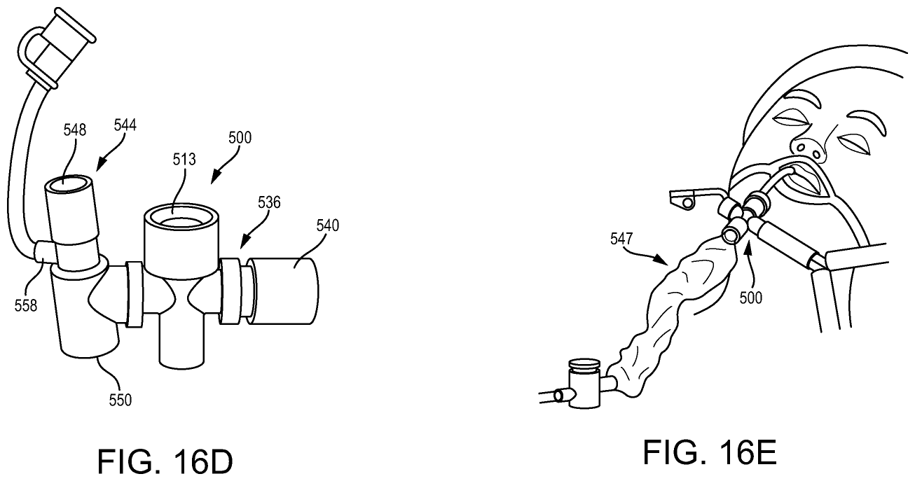

In some embodiments, a closed suction catheter system 547 may be detachably coupled to ventilator adapter 500 (e.g., via inlet 527) while the procedure module 544 is detached from airflow module 536, as shown in FIG. 16C. In some embodiments, ventilator adapter 500 may include a closed suction catheter system 547 secured to the airflow module 536 (e.g., via outlet 526), as shown in FIG. 16C, or the procedure module (e.g., via accessory port 548), as shown in FIGS. 16A and 16B. As shown in FIG. 16D, a fluid line can couple to the accessory port 558. FIG. 16E illustrates an example of the ventilator adapter 500 being used by a patient.

In some embodiments, the airflow module 536 may be connected to a closed suction catheter system 547, as shown in FIGS. 22A and 22B.

In some embodiments, the ventilation source may be changed from the ventilator connection port 540 to a resuscitation bag 514 via a resuscitation bag connection port 512 without disconnecting the ventilator adapter 500 or ventilator connection port 540. For example, a cap 513 may be removed from the resuscitation bag connection port 512 of ventilator adapter 500, and a resuscitation bag connector 515 included on the resuscitation bag 514 may be inserted into the resuscitation bag connection port 512, as shown in FIG. 23. The resuscitation bag connector 515 may be pushed into the resuscitation bag connection port 512 to engage the resuscitation bag piston 560. The resuscitation bag 514 may be ready for manual ventilation when the resuscitation bag connector 515 is inserted into the resuscitation bag connection port 512 until the resuscitation bag connector 515 is stopped by complete insertion and the resuscitation bag piston 560 is positioned at a bottom position. A filter may be placed between the resuscitation bag 514 and the ventilator adapter 500 to avoid cross-contamination.

Upon completion of a patient procedure, the resuscitation bag 514 may be disconnected. When the resuscitation bag 514 is removed, the resuscitation bag piston 560 may be in an up or first position and configured to disengage such that ventilator flow is redirected to the endotracheal tube 551.

In some embodiments, the ventilation source may be changed from the ventilator connection port 540 to a transport ventilator 517 without disconnecting the ventilator connection port 540. For example, a cap 513 may be removed from the resuscitation bag connection port 512 of ventilator adapter 500, (a tubing connection adapter or elbow may be needed). The transport ventilator connector 518 may be pushed into the resuscitation bag connection port 512 to engage the resuscitation bag piston 560. The transport ventilator 517 may be inserted into the resuscitation bag connection port 512 until the transport ventilator connector 518 is stopped by complete insertion and the resuscitation bag piston 560 is positioned at a bottom position. Such configuration may allow air to flow from the transport ventilator 517 through ventilator adapter 500 to the patient once the transport ventilator 517 is turned on. A cap 513 may be placed onto the disconnected ventilator connection port 540 on ventilator adapter 500, as shown in FIG. 24.

Upon completion of the transport or patient procedure, the cap 513 may be removed from the ventilator connection port 540 and a bedside or other ventilator may be connected to the ventilator connection port 540. When the ventilator is connected to the ventilator connection port 540 and is on, the resuscitation bag piston 560 may be in an up or first position and configured to disengage such that ventilator flow is directed from the ventilator connection port 540 to the endotracheal tube 551. A cap 513 may be placed onto the disconnected resuscitation bag connection port 512 on ventilator adapter 500, as shown in FIG. 24.

One or more methods for use of a respiratory valve apparatus 100 including a ventilator adapter 500 may include at least one or more of the following steps.

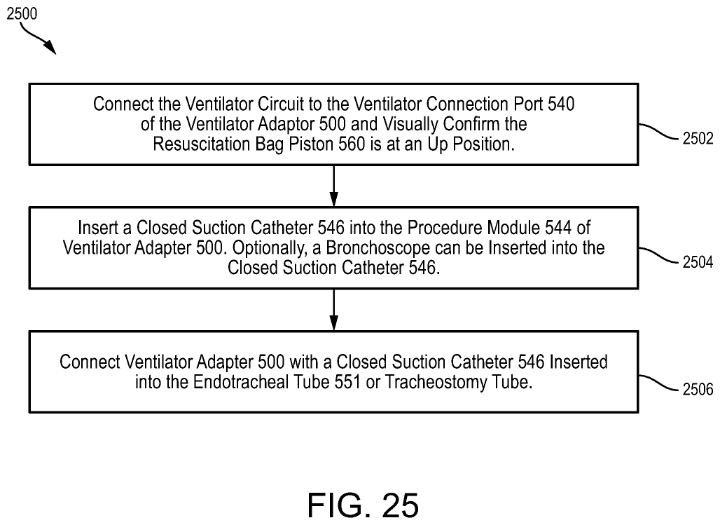

As shown in FIG. 25, an example method 2500 for connecting a ventilator circuit to ventilator connection port 540 with closed suction catheter 546 inserted into accessory port 558, may include one or more of the following steps. At a step 2502, a ventilator circuit may be attached to the ventilator connection port 540 of ventilator adaptor 500 and the resuscitation bag piston 560 may be visually confirmed to be in an up position. At a step 2504, a closed suction catheter 546 may be inserted into the procedure module 544 of ventilator adapter 500. Optionally, a bronchoscope can be inserted into the closed suction catheter 546. At a step 2506, ventilator adapter 500 may be connected with a closed suction catheter 546 inserted into the endotracheal tube 551 or tracheostomy tube.

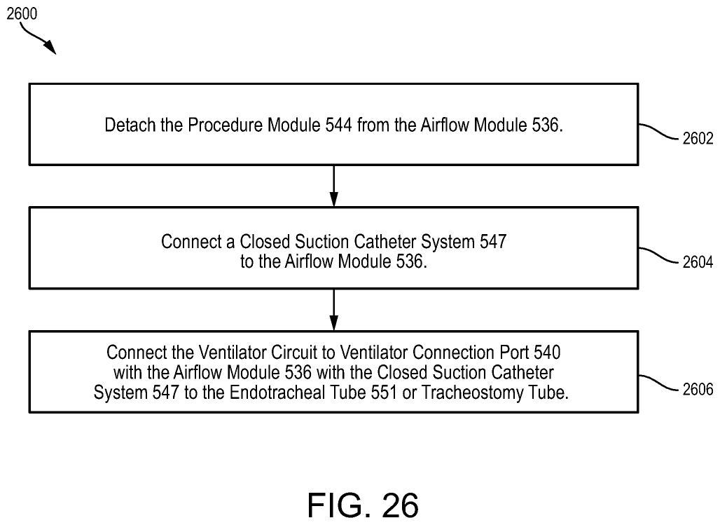

As shown in FIG. 26, a method 2600 for connecting a closed suction catheter system 547 to ventilator adapter 500 may include one or more of the following steps. At a step 2602, the procedure module 544 can be detached from the airflow module 536. At a step 2604, a closed suction catheter system 547 can be connected to the airflow module 536. At a step 2606, the airflow module 536 including the closed suction catheter system 547 may be connected to the endotracheal tube 551 or tracheostomy tube.

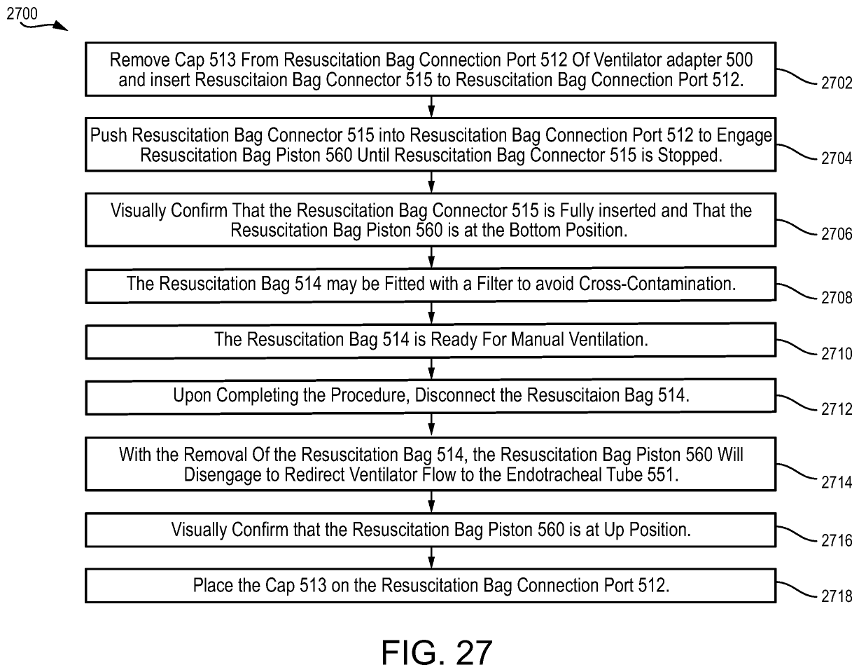

As shown in FIG. 27, a method 2700 for changing a ventilation source to a resuscitation bag 514 without disconnecting the ventilator adapter 500 may include one or more of the following steps. At a step 2702, the cap 513 may be removed from the resuscitation bag connection port 512 of ventilator adapter 500 and the resuscitation bag connector 515 may be inserted into the resuscitation bag connection port 512. At a step 2704, the resuscitation bag connector 515 may be pushed into the resuscitation bag connection port 512 to engage the resuscitation bag piston 560 until the resuscitation bag connector 515 is stopped. At a step 2706, the resuscitation bag connector 515 may be visually confirmed to be fully inserted and that the resuscitation bag piston 560 is at the bottom position. At a step 2708, the resuscitation bag 514 may be fitted with a filter configured to avoid cross-contamination.

At a step 2710, the resuscitation bag 514 may be ready for manual ventilation. At a step 2712, upon completing the procedure, the resuscitation bag 514 may be disconnected. At a step 2714, with the removal of the resuscitation bag 514, the resuscitation bag piston 560 may be disengaged to redirect ventilator flow to the endotracheal tube 551. At a step 2716, the resuscitation bag piston 560 may be visually confirmed to be in an up position. At a step 2718, the cap 513 may be placed back on the resuscitation bag connection port 512.

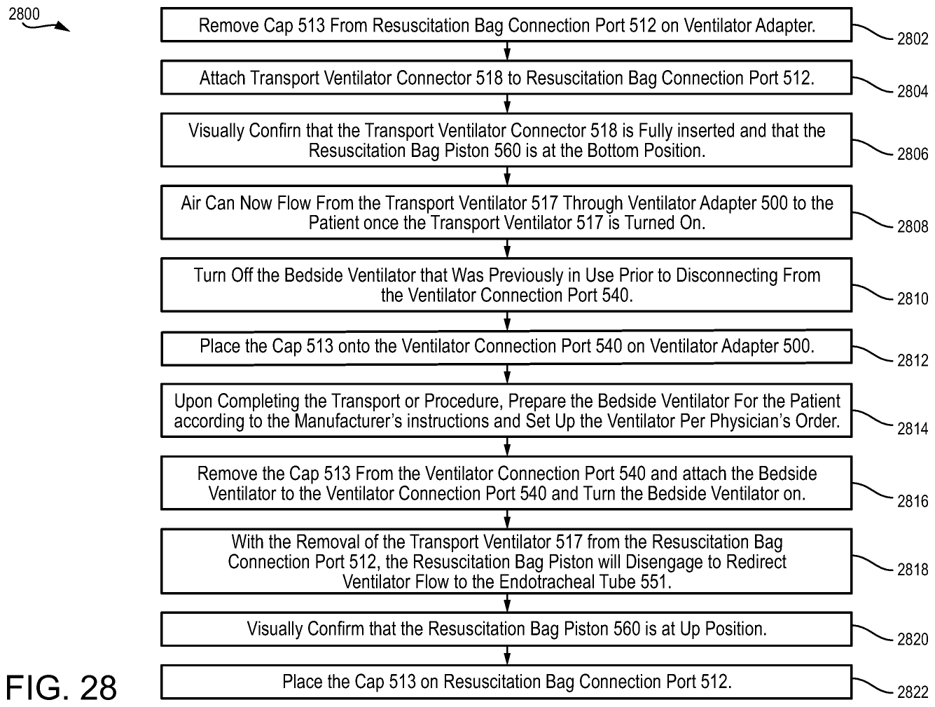

As shown in FIG. 28, a method 2800 for changing the ventilation source to a transport ventilator 517 without disconnecting the ventilator connection port 540 may include one or more of the following steps. At a step 2802, the cap 513 may be removed from the resuscitation bag connection port 512 on ventilator adapter 500 (a tubing connection adapter may be needed). At a step 2804, the transport ventilator connector 518 may be attached to the resuscitation bag connection port 512 and the transport ventilator connector 518 may be pushed into the resuscitation bag connection port 512 until it is stopped. At a step 2806, the transport ventilator connector 518 may be visually confirmed to be fully inserted and that the resuscitation bag piston 560 is at the bottom position. At a step 2808, air may flow from the transport ventilator 517 through ventilator adapter 500 to the patient once the transport ventilator 517 is turned on.

At a step 2810, the bedside ventilator that was previously in use may be turned off prior to disconnecting from the ventilator connection port 540. At a step 2812, the cap 513 may be placed onto the ventilator connection port 540 on ventilator adapter 500. At a step 2814, upon completing the transport or procedure, the bedside ventilator may be prepared for the patient according to the manufacturer's instructions. At a step 2816, the cap 513 may be removed from the ventilator connection port 540 and the bedside ventilator may be attached to the ventilator connection port 540 and turned on. At a step 2818, with the removal of the transport ventilator 517 from the resuscitation bag connection port 512, the resuscitation bag piston 560 may be disengaged to redirect ventilator flow to the endotracheal tube 551. At a step 2820, the resuscitation bag piston 560 may be visually confirmed to be in an up position. At a step 2822, the cap 513 may be placed on the resuscitation bag connection port 512.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The particular embodiments disclosed above are illustrative only, as the disclosure may be modified and practiced in different but equivalent manners apparent to those skilled in the art having the benefit of the teachings herein. For example, the process steps set forth above may be performed in a different order. Furthermore, no limitations are intended to the details of construction or design herein shown, other than as described in the claims below. It is therefore evident that the particular embodiments disclosed above may be altered or modified and all such variations are considered within the scope and spirit of the disclosure. Note that the use of terms, such as “first,” “second,” “third” or “fourth” to describe various processes or structures in this specification and in the attached claims is only used as a shorthand reference to such steps/structures and does not necessarily imply that such steps/structures are performed/formed in that ordered sequence. Of course, depending upon the exact claim language, an ordered sequence of such processes may or may not be required. Accordingly, the protection sought herein is as set forth in the claims below.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. A respiratory valve apparatus, comprising:
a first port configured to releasably couple to a ventilator;
a second port configured to releasably couple to a resuscitation bag or a transport ventilator;
a third port configured to releasably couple to an endotracheal tube; and
a piston slidably disposed within a housing of the respiratory valve apparatus, the piston being biased in a first position within the housing, the piston comprising:
a first flow pathway that allows fluid flow between the first port and the third port when the piston is in the first position; and
a second flow pathway that allows fluid flow between the second port and the third port when the piston is in a second position; and
wherein the piston prevents fluid flow between the second port and the third port when in the first position and prevents fluid flow between the first port and the third port when in the second position.

2. The respiratory valve apparatus of claim 1, further comprising a spring that biases the piston in the first position.

3. The respiratory valve apparatus of claim 2, wherein the spring is compressible to thereby allow the piston to move into a second position.

4. The respiratory valve apparatus of claim 3, wherein the piston is positioned adjacent the second port when in the first position for allowing the resuscitation bag or transport ventilator to move the piston into the second positon as a result of coupling the resuscitation bag or transport ventilator to the second port.

5. The respiratory valve apparatus of claim 4, wherein the movement of the piston into the second position prevents fluid flow through the first port and allows fluid flow through and between the second port and the third port.

6. The respiratory valve apparatus of claim 5, further comprising a first conduit including the first port at a first end and a first connection member at a second end, the first connection member being coupled to the housing and allowing rotational movement of the first conduit relative to the housing.

7. The respiratory valve apparatus of claim 6, further comprising a second conduit including the third port and a fourth port configured to releasably couple an accessory device.

8. The respiratory valve apparatus of claim 7, wherein the accessory device includes one or more of a suction catheter, a bronchoscope, and a drug delivery catheter.

9. The respiratory valve apparatus of claim 7, wherein the second conduit further includes a second connection member configured to couple to the housing and allow rotational movement of the second conduit relative to the housing.

10. The respiratory valve apparatus of claim 7, wherein the fourth port includes a sealing member that prevents fluid flow therethrough when the accessory device is uncoupled from the fourth port.

11. A respiratory valve apparatus, comprising:
a first port configured to releasably couple to a ventilator;
a second port configured to releasably couple to a resuscitation bag or a transport ventilator;
a third port configured to releasably couple to an endotracheal tube; and
a lever member pivotably disposed within a flow pathway extending along a housing of the respiratory valve apparatus, the lever member being biased in a first position to allow fluid flow between the first port and the third port, the lever member being positioned adjacent the second port, the lever member pivoting into a second position as a result of coupling the resuscitation bag or the transport ventilator to the second port thereby allowing fluid flow between the second port and the third port.

12. The respiratory valve apparatus of claim 11, wherein the lever member prevents fluid flow between the second port and the third port when the lever member is in the first position, the lever member prevents fluid flow between the first port and the third port when the lever member is in the second position.

13. A modular respiratory valve apparatus, comprising:
an airflow module comprising:
a first port configured to releasably couple to a ventilator;
a second port configured to releasably couple to a resuscitation bag or a transport ventilator; and
a first housing connection port; and
a procedure module comprising:
a third port configured to releasably couple to an endotracheal tube; and
a second housing connection port configured to releasably couple to the first housing connection port; and
a piston slidably disposed within the airflow module, the piston being biased in a first position within the airflow module, the piston comprising:
a first flow pathway that allows fluid flow between the first port and the third port when the piston is in the first position; and
a second flow pathway that allows fluid flow between the second port and the third port when the piston is in a second position; and
wherein the piston prevents fluid flow between the airflow module and the procedure module when in the second position and allows fluid flow between the first port and the third port when in the first position.

14. The modular respiratory valve apparatus of claim 13, further comprising a spring that biases the piston in the first position.

15. The modular respiratory valve apparatus of claim 14, wherein the spring is compressible to thereby allow the piston to move into a second position.

16. The respiratory valve apparatus of claim 15, wherein the piston is positioned adjacent the second port when in the first position for allowing the resuscitation bag or transport ventilator to move the piston into the second positon as a result of coupling the resuscitation bag or transport ventilator to the second port.

17. The respiratory valve apparatus of claim 16, wherein the movement of the piston into the second position prevents fluid flow through the first port and allows fluid flow through and between the second port and the third port.

18. The respiratory valve apparatus of claim 17, further comprising a first conduit including the first port at a first end and a first connection member at a second end, the first connection member being coupled to the housing and allowing rotational movement of the first conduit relative to the housing.

19. The respiratory valve apparatus of claim 18, further comprising a second conduit including the third port and a fourth port configured to releasably couple an accessory device.

20. The respiratory valve apparatus of claim 19, wherein the accessory device includes one or more of a suction catheter, a bronchoscope, and a drug delivery catheter.

21. The respiratory valve apparatus of claim 19, wherein the second conduit further includes a second connection member configured to couple to the housing and allow rotational movement of the second conduit relative to the housing.

22. The respiratory valve apparatus of claim 19, wherein the fourth port includes a sealing member that prevents fluid flow therethrough when the accessory device is uncoupled from the fourth port.

23. A method of a respiratory valve apparatus, comprising:
coupling, at a first port of the respiratory valve apparatus, a connecting end of a ventilator, the respiratory valve apparatus comprising:
a second port configured to releasably couple to a resuscitation bag or a transport ventilator;
a third port configured to releasably couple to an endotracheal tube; and
a piston slidably disposed within a housing of the respiratory valve apparatus, the piston being biased in a first position within the housing, the piston comprising:
a first flow pathway that allows fluid flow between the first port and the third port when the piston is in the first position; and
a second flow pathway that allows fluid flow between the second port and the third port when the piston is in a second position; and
coupling, at the third port of the respiratory valve apparatus, a connecting end of the endotracheal tube;
coupling, at the second port of the respiratory valve apparatus, a connecting end of the resuscitation bag or the transport ventilator,
wherein the coupling of the resuscitation bag or the transport ventilator to the second port forces the piston into a second position thereby allowing fluid flow between the second port and the third port and preventing fluid flow between the first port and the third port.

24. The method of claim 23, further comprising:
uncoupling the connecting end of the resuscitation bag or the transport ventilator from the second port thereby allowing the piston to move into the first position to allow fluid flow between the first port and the third port and prevent fluid flow between the second port and the third port.

25. The method of claim 23, wherein the respiratory valve apparatus further comprises a spring that biases the piston in the first position.

26. The method of claim 25, wherein the spring is compressible to thereby allow the piston to move into a second position.

27. The method of claim 26, wherein the piston is positioned adjacent the second port when in the first position for allowing the resuscitation bag or transport ventilator to move the piston into the second positon as a result of coupling the resuscitation bag or transport ventilator to the second port.

28. The method of claim 27, wherein the respiratory valve apparatus further comprises a second conduit including the third port and a fourth port configured to releasably couple an accessory device.

29. The method of claim 28, further comprising coupling one or more of a suction catheter, a bronchoscope, and a drug delivery catheter to the third port and/or the fourth port.

30. The method of claim 28, wherein the fourth port includes a sealing member that prevents fluid flow therethrough when the accessory device is uncoupled from the fourth port.

31. A method of a respiratory valve apparatus, comprising:
coupling, at a first port of the respiratory valve apparatus, a connecting end of a ventilator, the respiratory valve apparatus comprising:
a second port configured to releasably couple to a resuscitation bag or a transport ventilator;
a third port configured to releasably couple to an endotracheal tube; and
a lever member pivotably disposed within a flow pathway extending along a housing of the respiratory valve apparatus, the lever member being biased in a first position to allow fluid flow between the first port and the third port; and
coupling, at the third port of the respiratory valve apparatus, a connecting end of the endotracheal tube;
coupling, at the second port of the respiratory valve apparatus, a connecting end of the resuscitation bag or the transport ventilator,
wherein the coupling of the resuscitation bag or the transport ventilator to the second port forces the lever member to pivot into a second position thereby allowing fluid flow between the second port and the third port and preventing fluid flow between the first port and the third port.

32. The method of claim 31, further comprising:
uncoupling the connecting end of the resuscitation bag or the transport ventilator from the second port thereby allowing the lever member to pivot into the first position to allow fluid flow between the first port and the third port and prevent fluid flow between the second port and the third port.

33. The method of claim 32, wherein the lever member is positioned adjacent the second port when in the first position for allowing the resuscitation bag or transport ventilator to move the lever member into the second positon as a result of coupling the resuscitation bag or transport ventilator to the second port.

34. The method of claim 33, wherein the respiratory valve apparatus further comprises a second conduit including the third port and a fourth port configured to releasably couple an accessory device.

35. The method of claim 34, further comprising coupling one or more of a suction catheter, a bronchoscope, and a drug delivery catheter to the third port and/or the fourth port.

36. The method of claim 35, wherein the fourth port includes a sealing member that prevents fluid flow therethrough when the accessory device is uncoupled from the fourth port.
